(19) United States
(12) Patent Application Publication
(10) Pub. No.: US 2023/0235326 A1
(43) Pub. Date: Jul. 27, 2023

(54) METHODS AND PHARMACEUTICAL COMPOSITIONS FOR TREATING OCULAR DISEASES

(71) Applicants: INSERM (INSTITUT NATIONAL DE LA SANTÉ ET DE LA RECHERCHE MÉDICALE), Paris (FR); UNIVERSITÉ PARIS CITÉ, Paris (FR); FONDATION IMAGINE, Paris (FR); ASSISTANCE PUBLIQUE-HÔPITAUX DE PARIS (APHP), Paris (FR)

(72) Inventors: Jean-Michel ROZET, Paris (FR); Lucas FARES TAIE, Paris (FR); Brigitte NEDELEC, Paris (FR); Clémentine ANGEE, Paris (FR); Josseline KAPLAN, Paris (FR)

(21) Appl. No.: 18/000,538

(22) PCT Filed: Jun. 4, 2021
(86) PCT No.: PCT/EP2021/064988

§ 371 Date: Dec. 2, 2022

(30) Foreign Application Priority Data
Jun. 5, 2020 (EP) 20305596.7

(51) Int. Cl. C12N 15/113; C07K 16/18; C12N 15/115; A61P 27/02

(52) U.S. Cl. CPC C12N 15/113; C07K 16/18; C12N 15/115; A61P 27/02; C12N 2310/14; C12N 2310/141; C12N 2310/531; C12N 2310/11; C12N 2310/13; C12N 2310/12

(57) ABSTRACT

The present invention relates to a method for treating ocular disease in a subject in need thereof comprising a step of administering to said subject a therapeutically amount of an inhibitor of SOX21 gene expression and/or activity. By studying a mouse model of congenital microcoria, the inventors demonstrate that this ultra-rare and purely ocular disease is due to unanticipated complex mechanisms linked with 3D regulation of gene expression. They propose that the disease is due to the illegitimate expression of a transcription factor, SOX21, induced by the adoption of a DCT enhancer(s). They show that SOX21 binds to a regulatory region of the Tgfβ2 gene and the inventors demonstrate overexpression of this trophic factor in the iris and accumulation of its product in the aqueous humor of the mouse carrying the minimal MCOR deletion which recapitulates the observed accumulation in patients with POAG and one of our patient with MCOR.

### FIELD OF THE INVENTION

The invention is in the field of ophthalmology. More particularly, the invention relates to methods and pharmaceutical compositions to treat ocular disease, including glaucoma, primary open angle glaucoma and myopia.

### BACKGROUND OF THE INVENTION

The iris and ciliary body (CB) are continuous ocular tissues organized into three main layers (from the visible surface to the posterior part next to the lens): stroma, anterior epithelium layer (AEL) and posterior epithelium layer (PEL). The root of the iris is attached to the CB and the corneal-sclera junction, creating an empty space known as the irido-corneal angle. The eye contains a fluid known as aqueous humor that provides nourishment to its structures. This fluid is produced by the CB. It flows between the iris and lens, through the pupil to the anterior part of the iris where is drained out in the Schlemm's canal after passing through a sieve-like structure called the trabecular meshwork (TM), located at the merging of the cornea-sclera with the iris root (Davis & Ashery-Padan, 2008). Iris and CB integrity is required to regulate both, the amount of light reaching the retina and the intraocular pressure (TOP), increase of which is a major risk factor for the optic nerve damage (glaucoma, GLC) (Gould et al., 2004). Congenital microcoria (MCOR) is an ultra-rare autosomal dominant malformation of the iris which affects both these functions. It is characterized by partial or complete absence of iris dilator muscle that normally develops from the iris AEL and extend longitudinally in the stroma from the iris root to pupillary margin. The sphincter muscle within the stroma, near the pupil margin, functions normally and can constrict the pupil. The dilator muscle anomaly manifests in pinhole pupils (<2 mm) that dilate poorly or not at all, and iris transillumination (Simpson & Parsons, 1989). A marked elongation of the axial length of the eye (axial myopia) and open angle GLC are noted in 80% and 30% individuals affected with MCOR, respectively (Toulemont et al., 1995). Myopia is the most common eye condition and GLC the second leading cause of blindness worldwide, affecting 70 million people (3.54% of the world population in 2020) (Tham et al., 2014). Primary open angle glaucoma (POAG) makes up the majority of GLC cases (3.05% of the world population in 2020) (Tham et al., 2014).

The elongation of the axial length of the eye typically leads to high myopia in MCOR. It could be due to early visual deprivation (form-deprivation myopia) elicited by a propensity to close eyelids to reduce glare caused by iris transillumination (Toulemont et al., 1995). Ocular occlusion can indeed cause axial myopia as demonstrated by monoocular myopia in infants with unilateral eyelid closure and surgical eyelid closure at various time of postnatal development in experimental animal models of myopia (Weiss, 2003). Elevated IOP during postnatal eye growth could also increase the length of the eye (Toulemont et al., 1995). However, the lack of correlation between myopia and oculo-cutaneous albinism where iris transillumination and photoaversion are major symptoms, challenges these assumptions, as does the absence of increased axial myopia in MCOR individuals with GLC compared to those with normal IOP.

GLC in congenital microcoria is characterized by an early onset (mean age at diagnosis 20±10 years) (Sergouniotis et al., 2017; Tawara et al., 2005; Toulemont et al., 1995) and a high IOP. Glaucomatous damages on the optic nerve are difficult to monitor due to pupillary miosis which complicates fundus examination, and severe myopia which modifies the shape of the optic disc (Toulemont et al., 1995). Affected individuals typically end-up blind around their fourth-fifth decades. The irido-corneal angle of MCOR individuals is open and typically displays a normal aspect (Bremner et al., 2004; Ramirez-Miranda et al., 2011; Simpson & Parsons, 1989) (open angle GLC, OAG). Occasionally, there exist an abnormal insertion of the root of the iris into the scleral spur; this anomaly does not close the angle (Mazzeo et al., 1986; Tawara et al., 2005; Toulemont et al., 1995). Some authors have considered connecting this irido-corneal angle anomaly to GLC but such a link is spurious, especially in the view of the higher prevalence of the angle anomaly in MCOR individuals with normal IOP, even at advanced ages, than in MCOR cases with OAG (Mazzeo et al., 1986; Sergouniotis et al., 2017; Toulemont et al., 1995). In the absence of precise knowledge of the causes of OAG, the term of childhood-onset or juvenile GLC (Ramprasad et al., s. d.; Rouillac et al., 1998) should be preferred to the terms proposed by some which associate GLC to the malformation (dysgenesis) of the iridocorneal angle, i.e. dysgenesic GCL (Coulon et al., 1986) or developmental GLC (Tawara et al., 2005).

The identification of submicroscopic chromosome 13q32.1 rearrangements in all tested MCOR families (Fares-Taie et al., 2015; Pozza et al., 2020; Sergouniotis et al., 2017), has involved 3D deregulation of gene expression as the cause of the disease but the mechanisms underlying the developmental failure of the iris and whether OAG and myopia are effectively consequences of the iris malformation remains a mystery.

Thus, there is a need to characterize the molecular mechanisms underlying iris malformation, OAG and high myopia in MCOR patients and to determine whether OAG and high myopia are secondary to the abnormal development of the iris or independent features.

### SUMMARY OF THE INVENTION

The invention relates to a method for treating ocular disease in a subject in need thereof comprising a step of administering to said subject a therapeutically amount of an inhibitor of SOX21 gene expression and/or activity. In a particular, the invention is defined by claims.

### DETAILED DESCRIPTION OF THE INVENTION

By studying a mouse model the Inventors generated, which carries the critical MCOR-causing deletion (Fares-Taie et al., 2015), they have suggested that this ultra-rare and purely ocular disease is due to unanticipated complex mechanisms linked with 3D regulation of gene expression (FIGS. 1A,B,C and 3A,B). Their data indicate that the disease is due to the illegitimate expression of a transcription factor, SOX21, in the iris and the ciliary body, possibly induced by the adoption of DCT enhancer(s). Using a combination of CHIPSeq, RNAseq and ELISA they have shown that (i) SOX21 binds to a regulatory region of the Tgfβ2 gene (FIG. 2A-B) (ii) Tgfβ2 mRNA is overexpressed in the iris (data not shown) and its product (TGFβ2) accumulates in the aqueous humor of the MCOR mouse model (FIG. 3A), which recapitulates the observed accumulation in a human MCOR individual (FIG. 3B) and individuals affected with common primary open angle glaucoma (POAG)(Carreon et al., 2017; Vranka et al., 2015).

The elevation of TGFB2 in the aqueous humor has been shown to induce extracellular matrix (ECM) accumulation in the deepest portion of the TM adjacent to the Schlemm's canal known as the juxtacanalicular region in human and mouse. ECM accumulation is a main trigger of OAG, including POAG, by reducing the filtering capacity of the TM. Increased aqueous humor outflow resistance leads to elevation of the IOP and glaucoma, that is to say the death of the retinal ganglion cells, the axons of which form the optic nerve (Carreon et al., 2017; Vranka et al., 2015). Accordingly, the Inventors have shown decreased abundance of glial cells in the optic nerve of the MCOR mouse model (FIG. 3C), strongly supporting glaucomatous optic nerve degradation.

Together, the Inventors' results support the view that OAG in MCOR is not a consequence of the irido-corneal anomaly, but rather it seems to be a direct consequence of TGFβ2 overexpression as is POAG. Furthermore, knowing that TGFβ2 may act as a critical factor in axial elongation of the eye globe (Jia et al., 2017), its overexpression could also account for high myopia in MCOR.

Accordingly, their data disclose a novel pathway of TGFβ2 regulation which involves SOX21 as a potential therapeutic target for OAG both in MCOR and POAG.

### Method for Treating Ocular Disease

Accordingly, in a first aspect, the invention relates to a method for treating ocular disease in a subject in need thereof comprising a step of administering to said subject a therapeutically effective amount of an inhibitor of SOX21 gene expression and/or activity.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “ocular disease” refers to a disease association with eyes. In the context of the invention, the ocular disease is related to an increase of TGFβ2 expression and/or activity. More particularly, the ocular disease is related to a dysfunction and/or anatomy anomaly of iris such as: elongation of the axial length of the eye, increase of intraocular pressure (TOP), malformation of the iris etc In a particular embodiment, the ocular disease is selected in the following group consisting of but not limited to: Congenital microcoria (MCOR), glaucoma, open angle glaucoma (AOG) or myopia.

In a particular embodiment, the ocular disease is congenital microcoria.

As used herein, the term “Congenital microcoria” (MCOR) is an ultra-rare hereditary disease of iris development transmitted as an autosomal dominant trait. It is characterized by partial or total absence of dilator muscle (DM) fibers. The DM developmental anomaly manifests in pinhole pupils (<2 mm) that dilate poorly or not at all, and iris transillumination. Axial myopia and OAG are noted in 80% and 30% individuals affected with MCOR, respectively.

In a particular embodiment, the ocular disease is glaucoma.

As used herein, the term “glaucoma” refers to a group of eye diseases encompassing a broad spectrum of clinical presentations, etiologies, and treatment modalities. Glaucoma causes pathological changes in the optic nerve, visible on the optic disk, and it causes corresponding visual field loss, resulting in blindness if untreated. Lowering intraocular pressure is the major treatment goal in all glaucomas. Glaucoma is broadly classified into two categories: closed-angle glaucoma, also known as angle closure glaucoma, and open-angle glaucoma. Closed-angle glaucoma is caused by closure of the anterior chamber angle by contact between the iris and the inner surface of the trabecular meshwork. Closure of this anatomical angle prevents normal drainage of aqueous from the anterior chamber of the eye. Open-angle glaucoma is any glaucoma in which the exit of aqueous through the trabecular meshwork is diminished while the angle of the anterior chamber remains open.

In a particular embodiment the glaucoma is open angle glaucoma (OAG).

As used herein, the term open angle glaucoma (AOG) refers to one of glaucoma caused by the slow clogging of the drainage canals, resulting in increased intraocular pressure and optic nerve degradations. “Open-angle” means that the angle where the iris meets the cornea also known as the irido-corneal or anterior chamber angle, is as wide and open as it should be. OAG develops insidiously and is a lifelong condition. Open-angle glaucoma is also called primary (POAG) or chronic glaucoma. It occurs in 30% of the MCOR patients. OAG is the second leading cause of blindness worldwide⁵, affecting 70 million people worldwide.

In a particular embodiment, the ocular diseases is myopia.

As used herein, the term “myopia” refers to one of myopia caused by the elongation of the axial length of the eye globe (axial myopia). Myopia is the most common eye condition⁶.

In a particular embodiment, the ocular disease as described above is found in children and young adult.

In another embodiment, the ocular disease as described above is found in an elderly person.

As used herein, the term “subject” denotes a mammal, such as a rodent, a feline, a canine, and a primate. Particularly, the subject according to the invention is a human. More particularly, the subject is suffering or susceptible to suffer from an ocular disease as described above. In a particular embodiment, the subject is suffering or susceptible to suffer MCOR. In a particular embodiment, the subject is suffering or susceptible to suffer from glaucoma. In a particular embodiment, the subject is suffering or susceptible to suffer from open angle glaucoma (OAG). In a particular embodiment, the subject is suffering or susceptible to suffer from myopia. In a particular embodiment, the subject has or is susceptible to have an over expression and/or activity of TGFβ2. In another embodiment, the subject is a child or a young adult. In another embodiment, the subject is an elderly person.

The results of the inventors are consistent with the view that SOX21-mediated TGFβ2 overexpression is the trigger that causes in an independent manner the DM malformation, OAG and myopia in individuals carrying genomic rearrangements at the MCOR locus.

Accordingly, the invention relates to use of an inhibitor of SOX21 expression and/or activity in the treatment of ocular disease.

More particularly, the inhibitor according to the invention is suitable to treat ocular disease selected from the group consisting of but not limited to: MCOR, glaucoma, OAG, myopia.

As used herein, the term “SOX21” refers to SRY-Box Transcription Factor 21. It is a protein that in humans encoded by the SOX21 gene. It is a member of the SOX gene family of transcription factors. SOX genes encode a family of transcription factors that bind to the minor groove in DNA, and belong to a super-family of genes characterized by a homologous sequence called the HMG-box (for high mobility group). This HMG box is a DNA binding domain that is highly conserved throughout eukaryotic species. SOX21 is a 276 amino acid residue protein that has an N-terminal HMG-box and a C-terminal domain that is required for the SOX21 neurogenesis function. Human and mouse SOX21 share 99% amino acid sequence identity. The only known function of SOX21 in the eye comes from studies in the chick and zebrafish (Lan et al., 2011; Uchikawa et al., 1999). In the chick, SOX21 is transiently activated during the early phases of optic vesicle morphogenesis and specification in the lens and retina but no longer expressed afterwards (Uchikawa et al., 1999). The ocular expression of SOX21 stops before the iris starts developing. Its loss-of-function in the chick, as in zebrafish, interferes with normal lens development (Pauls et al., 2012).

The naturally occurring human SOX21 gene has a nucleotide sequence as shown in Genbank Accession number NM_007084 and the naturally occurring human SOX21 protein has an amino acid sequence as shown in Genbank Accession numbers NP 009015. The naturally occurring mouse SOX21 gene has a nucleotide sequence as shown in Genbank Accession number NM_177753 and the naturally occurring mouse SOX21 protein has an amino acid sequence as shown in Genbank Accession numbers NP_808421.

As used herein, the term “gene” has its general meaning in the art and refers to means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed.

As used herein the “allele” has its general meaning in the art and refers to an alternative form of a gene (one member of a pair) that is located at a specific position on a specific chromosome which, when translated result in functional or dysfunctional (including nonexistent) gene products.

As used herein, the term “protein” has its general meaning in the art and refers to one or more long chains of amino acid residues which comprise all or part of one or more proteins or enzymes.

The human SOX21 has the following nucleotide sequence in the art SEQ ID NO:1:

| | |
|---|---|
| 1 | AAACACTCCA GCCGCTGAGA GCCCCCTTTG |
| | GCACTTGGCA GCACGCGGCG GCGGGCTCCT |
| 61 | CGGCTCAACT TCGAGGAGTC TCCGCGACGC |
| | AACTTTTGGG GACGCTTTGC ATTTAAGAGA |
| 121 | GAACGACCGA GGAGGAGGAG CGCTCTGCCC |
| | GGCCGCCGCT ACCTGCGGGG AGCTCACCAG |
| 181 | CAAACGCCAC TGCAGACGAA GGACCCAAAG |
| | AACGTAAAGG GCAAACTGCC GCCGCGGGGA |
| 241 | GGGGGCACCG CCGAGAAGTT AGAGTGTCCC |
| | AGAGACAACC TGCTCGAGCG CTCGGCCGGA |
| 301 | GACACTAAGG CGGCCCGGGG CGCGGCGTGG |
| | CCCTGGGCTG GTCCCCCAGC CCCCTCCTCC |
| 361 | GGGGCGGGAG CGACGCCGGG GCGCGACGAG |
| | CCCCGGCCGG CCGAGCGGGT CTCCGCGGGC |
| 421 | AGCCAACATT GATTTCCTCC GGGCCGAGGG |
| | CGAGGGCCCG GGAGGCGGCG GGCTGCAGCC |
| 481 | GCGGCAGGGC GAGAGCATGT CCAAGCCGGT |
| | GGACCACGTC AAGCGGCCCA TGAACGCCTT |
| 541 | CATGGTGTGG TCGCGGGCTC AGCGGCGCAA |
| | GATGGCCCAG GAGAACCCCA AGATGCACAA |
| 601 | CTCGGAGATC AGCAAGCGCT TGGGCGCCGA |
| | GTGGAAACTG CTCACAGAGT CGGAGAAGCG |
| 661 | GCCGTTCATC GACGAGGCCA AGCGTCTACG |
| | CGCCATGCAC ATGAAGGAGC ACCCCGACTA |
| 721 | CAAGTACCGG CCGCGGCGCA AGCCCAAGAC |
| | GCTGCTCAAG AAGGACAAGT TCGCCTTCCC |
| 781 | GGTGCCCTAC GGCCTGGGCG GCGTGGCGGA |
| | CGCCGAGCAC CCTGCGCTCA AGGCGGGCGC |
| 841 | CGGGCTGCAC GCGGGGGCGG GCGGCGGCCT |
| | GGTGCCTGAG TCGCTGCTCG CCAATCCCGA |
| 901 | GAAGGCGGCC GCGGCCGCCG CCGCTGCCGC |
| | CGCACGCGTC TTCTTCCCGC AGTCGGCCGC |
| 961 | TGCCGCCGCC GCTGCCGCCG CCGCCGCCGC |
| | CGCGGGCAGC CCCTACTCGC TGCTCGACCT |
| 1021 | GGGCTCCAAA ATGGCAGAGA TCTCGTCGTC |
| | CTCGTCCGGC CTCCCGTACG CGTCGTCGCT |
| 1081 | GGGCTACCCG ACCGCGGGCG CGGGCGCCTT |
| | CCACGGCGCG GCGGGGGCGG CTGCAGCGGC |
| 1141 | GGCCGCCGCC GCCGGGGGGC ACACGCACTC |
| | GCACCCCAGC CCGGGCAACC CGGGCTACAT |
| 1201 | GATCCCGTGC AACTGCAGCG CGTGGCCCAG |
| | CCCCGGGCTG CAGCCGCCGC TCGCCTACAT |
| 1261 | CCTGCTGCCG GGCATGGGCA AGCCCCAGCT |
| | GGACCCCTAC CCCGCGGCCT ACGCTGCCGC |
| 1321 | GCTATGACCC CGCGGGGCCG CCTCGCGAGG |
| | ACCGGTGTGC ACACGTGTAC ATATGTATAG |
| 1381 | GTACGAGCGC TGCGGCCTCC CCGTGCGCCC |
| | TCCCGCGACC GGGGGCCCGG TTTGTATGTA |
| 1441 | CATAGAATGT ATAGGTGCCA GGTAGAGGCA |
| | GAGAGGCCAG GCGGGGCAGG AGTGGCCAAG |
| 1501 | CGCGCAAGGG CGCGGGCGAG CAGGCCTGTG |
| | AATTCGCAGG ATCATTTCAG ACCCGCACTT |
| 1561 | CGGCAGCCAA CTCGAAAGCA GGCGGTTGTG |
| | TGCGGCAGCA GTTGGCGTTT GCTTTGCACT |
| 1621 | TCGGAACCTG TTGCGTTTTG ACCCACGGAG |
| | GTGGAGGAGT AACTTTTTGA CATGTTGGCC |
| 1681 | TTTCCAGTTT TGTTGGAAGT TTCATGGTCG |
| | GTTTTGTTTT TGTTTCTCAT TCTTCTTCCT |
| 1741 | CGCCCCTCAG CCCCCCAACC CCCAACCCCC |
| | TCCCGGTCCG TGTTGCATGC ACGCTGTTCA |
| 1801 | AATGTGAGGT CTGAAATGGC TGGCACACGG |
| | GAAAAGCTGC TTGTGTCATT CGTTTCTGGG |
| 1861 | AGTGGGATGG CTCTGAGCAG CCTCGCCTCC |
| | CTGTTTGTAC TATTTGAACT TTGCAGATCT |
| 1921 | CTGTTCTCTC AAGCAGAACT CCCAACCAGA |
| | TCCATTCTTG ACCAGTGACC GGCTCGAATC |
| 1981 | TGGCCTTTTG TGTGAGATGA TCACGGTTTC |
| | TTTTGTTTAT CACGCCATTT GCAAATCAGA |
| 2041 | GCAAGAGCTC TTTCTCAAGG GCAAGAAACG |
| | CAAACAAGAA ATATTTGTGA GATGAAAGTT |
| 2101 | GTCAATTGGA TTTTCTTCCT AAACAAACAA |
| | CAACAACAAA CTACTAGAAG TCTCCCTGAG |
| 2161 | TCCACTCGCT TGGATTTCTG ACACAGTTTA |
| | CAAAAAAGGA AAAAGGCACT GCTCCTATTT |
| 2221 | TCCCTTATGG CTGAGTTCAC CTTAAGATTG |
| | TAAATGTGTA TATGTCAGTG AAAACATTGA |
| 2281 | GGCTTGGAAA ATGTGTTATT TTCGTTGCCC |
| | TAAGTTTGAG TCGACTTTAG ACTCAAAAAC |
| 2341 | ATTTTGAGCG AATATCAAAG TTAACTTTTA |
| | AAAATTGCGA AACTATTTCA GAATCGCAAT |
| 2401 | TTTATCGAAG ATTAAATCAG ACTTTTTTGT |
| | CTGGTAATTA TATATTTATT ATTTAGCAAA |
| 2461 | ACTGAAGAAA AAAAGCACAG AATTGTTTCA |
| | ACAGATGTCT CTCATTTTCA GCTAGCATTT |
| 2521 | CTCTCCCAAG TTGAGCTGGT TTAATGTGTT |
| | TTGGATTTCC CTCCTCAATT GGCTTATTTT |
| 2581 | TTAGATCACC TGCAATTCAT TTGCAAATTG |
| | CAATAAAACA CATTTTAGAA AAAAGGAACC |
| 2641 | TTCAATTATT AGCTTTGTTT CTTTTTAAAT |
| | GTATATATTT TGACTAATGT TTGTGAATGA |
| 2701 | AGTTGGCTAA CATGTATTTA GTTTCATTTT |
| | GGCTTTATGT AATATAAAGT TTTTAAAATT |
| 2761 | TTAAATATGG TTTTAACCTT TATGTGTAAA |
| | TGATTTTCTA GTGTGACCTT CTAATTTAAT |
| 2821 | ATTAGACGTC TAAGGTATAT CTGTAAATTA |
| | GAATCCGACT ATCACTCTGT TCATTTTTTT |
| 2881 | TGAACAAAGA GTTTAAATAA AGCCTGAACC |
| | AGGGAAAAGA AAAA |

The human SOX21 has the following amino acid sequence in the art SEQ ID NO:2:

MSKPVDHVKRPMNAFMVWSRAQRRKMAQENPKMHNSEI
SKRLGAEWKLLTESEKRPFIDEAKRLRAMHMKEHPDYK
YRPRRKPKTLLKKDKFAFPVPYGLGGVADAEHPALKAG
AGLHAGAGGGLVPESLLANPEKAAAAAAAAAARVFFPQ
SAAAAAAAAAAAAAGSPYSLLDLGSKMAEISSSSSGLP
YASSLGYPTAGAGAFHGAAAAAAAAAAAAGGHTHSHPS
PGNPGYMIPCNCSAWPSPGLQPPLAYILLPGMGKPQLD
PYPAAYAAAL

As used herein, the term “inhibitor” refers to a natural or synthetic compound able to inhibit the activity and/or expression of SOX21 and/or its product. As used herein, the term “SOX21 expression” refers to SOX21 gene encodes a SOX21 protein (the SOX21 gene product). More particularly, the inhibition of SOX21 expression leads to a deregulation of the TGFb2 gene and/or its product TGFβ2. As used herein the terms “SOX21 activity” refers to its coordination with other genes such as SOX2 or PAX6 in the eye development and/or functioning. More particularly, the inhibition of SOX21 activity leads to a deregulation TGFβ2 gene, TGFβ2 protein or TGFβ2-signalling. Typically, in the context of the invention, the inhibition of SOX21 leads a decrease of TGFβ2 expression and/or activity.

In a particular embodiment, the inhibitor of SOX21 is an inhibitor of SOX21 expression.

An “inhibitor of SOX21 expression” refers to a natural or synthetic compound that has a biological effect to inhibit or significantly reduce the expression of the gene encoding for SOX21. Typically, the inhibitor of SOX21 expression has a biological effect on one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

In a particular embodiment, the method according to the invention, wherein the inhibitor of SOX21 gene expression is shRNA, siRNA, miRNA, antisense oligonucleotide, transcription factor decoy, ribozyme or an endonuclease.

In a particular embodiment, the inhibitor of SOX21 expression is a shRNA. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound.

In some embodiments, the inhibitor of SOX21 expression is a small inhibitory RNAs (siRNAs). SOX21 expression can be reduced by contacting the subject or cell with a small double stranded RNA (dsRNA), or a vector or construct causing the production of a small double stranded RNA, such that SOX21 expression is specifically inhibited (i.e. RNA interference or RNAi). Methods for selecting an appropriate dsRNA or dsRNA-encoding vector are well known in the art for genes whose sequence is known (e.g. see (Brummelkamp et al., 2002; Elbashir et al., 2001; Hannon, 2002; McManus et al., 2002; Tuschl et al., 1999) U.S. Pat. Nos. 6,573,099 and 6,506,559; and International Patent Publication Nos. WO 01/36646, WO 99/32619, and WO 01/68836). In a particular embodiment, the siRNA is ALN-PCS02 developed by Alnylam (phase 1 ongoing).

In some embodiments, the inhibitor of SOX21 expression is a miRNA. As used herein, the term “miRNAs” refers to mature microRNA (non-coding small RNAs) molecules that are generally 21 to 22 nucleotides in length, even though lengths of 19 and up to 23 nucleotides have been reported. miRNAs are each processed from longer precursor RNA molecules (“precursor miRNA”: pri-miRNA and pre-miRNA). Pri-miRNAs are transcribed either from non-protein-encoding genes or embedded into protein-coding genes (within introns or non-coding exons). The “precursor miRNAs” fold into hairpin structures containing imperfectly base-paired stems and are processed in two steps, catalyzed in animals by two Ribonuclease III-type endonucleases called Drosha and Dicer. The processed miRNAs (also referred to as “mature miRNA”) are assembled into large ribonucleoprotein complexes (RISCs) that can associate them with their target mRNA in order to repress translation.

In some embodiments, the inhibitor of SOX21 expression is an antisense oligonucleotide. Anti-sense oligonucleotides, including anti-sense RNA molecules and anti-sense DNA molecules, would act to directly block the translation of SOX21 mRNA by binding thereto and thus preventing protein translation or increasing mRNA degradation, thus decreasing the level of SOX21 proteins, and thus activity, in a cell. For example, antisense oligonucleotides of at least about 15 bases and complementary to unique regions of the mRNA transcript sequence encoding SOX21 can be synthesized, e.g., by conventional phosphodiester techniques and administered by e.g., intravenous, subcutaneous or intravitreal injection. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

The AON can be synthesized de novo using any of a number of procedures well known in the art. For example, the b-cyanoethyl phosphoramidite method (Beaucage et al., 1981); nucleoside H-phosphonate method (Garegg et al., 1986; Froehler et al., 1986, Garegg et al., 1986, Gaffney et al., 1988). These chemistries can be performed by a variety of automated nucleic acid synthesizers available in the market. These nucleic acids may be referred to as synthetic nucleic acids. Alternatively, AONs can be produced on a large scale in plasmids (see Sambrook, et al., 1989). AONs can be prepared from existing nucleic acid sequences using known techniques, such as those employing restriction enzymes, exonucleases or endonucleases. AONs prepared in this manner may be referred to as isolated nucleic acids.

The AON may be or are stabilized. A “stabilized” AON refers to an AON that is relatively resistant to in vivo degradation (e.g. via an exo- or endo-nuclease). Stabilization can be a function of length or secondary structure. Alternatively, AON stabilization can be accomplished via phosphate backbone modifications. Preferred stabilized AONs of the instant invention have a modified backbone, e.g. have phosphorothioate linkages to provide maximal activity and protect the AON from degradation by intracellular exo- and endo-nucleases. Other possible stabilizing modifications include phosphodiester modifications, combinations of phosphodiester and phosphorothioate modifications, methylphosphonate, methylphosphorothioate, phosphorodithioate, p-ethoxy, and combinations thereof. Chemically stabilized, modified versions of the AONs also include “Morpholinos” (phosphorodiamidate morpholino oligomers, PMOs), 2′-O-Met oligomers, 2′-Fluoro (2′-F) oligomers, tricyclo (tc)-DNAs, U7 short nuclear (sn) RNAs, tricyclo-DNA-oligoantisense molecules (U.S. Provisional Patent Application Ser. No. 61/212,384 For: Tricyclo-DNA Antisense Oligonucleotides, Compositions and Methods for the Treatment of Disease, filed Apr. 10, 2009, the complete contents of which is hereby incorporated by reference), unlocked nucleic acid (UNA), peptide nucleic acid (PNA), serinol nucleic acid (SNA), twisted intercalating nucleic acid (TINA), anhydrohexitol nucleic acid (HNA), cyclohexenyl nucleic acid (CeNA), D-altritol nucleic acid (ANA) and morpholino nucleic acid (MNA) have also been investigated in splice modulation. Recently, nucleobase-modified AOs containing 2-thioribothymidine, and 5-(phenyltriazol)-2-deoxyuridine nucleotides have been reported to induce exon skipping (Chen S, Le B T, Chakravarthy M, Kosbar T R, Veedu R N. Systematic evaluation of 2′-Fluoro modified chimeric antisense oligonucleotide-mediated exon skipping in vitro. Sci Rep. 2019 Apr. 15; 9(1):6078.)

In a particular embodiment, the antisense oligonucleotides may be 2′-O-Me RNA/ENA chimera oligonucleotides (Takagi M, Yagi M, Ishibashi K, Takeshima Y, Surono A, Matsuo M, Koizumi M. Design of 2′-O-Me RNA/ENA chimera oligonucleotides to induce exon skipping in dystrophin pre-mRNA. Nucleic Acids Symp Ser (Oxf). 2004; (48):297-8).

In another particular embodiment, the antisense oligonucleotides of the invention are LNA gapmers, i.e. chimeric antisense oligonucleotides that contains a central block of deoxynucleotide monomers sufficiently long to induce RNase H cleavage (Hagedorn P H, Persson R, Funder E., Albæk N, Dieme S L, Hansen D J, Møller M R, Papargyri N, Christiansen H, Hansen B R, Hansen H F, Jensen M A, Koch T. Locked nucleic acid: modality, diversity, and drug discovery. Drug Discovery Today Volume 23, Issue 1, January 2018, Pages 101-114).

In another particular embodiment, the antisense oligonucleotides of the invention are 2′-O-methyl-phosphorothioate nucleotides.

Other forms of AONs that may be used to this effect are AON sequences coupled to small nuclear RNA molecules such as U1 or U7 in combination with a viral transfer method based on, but not limited to, lentivirus or adeno-associated virus (Denti, M A, et al, 2008; Goyenvalle, A, et al, 2004).

In some embodiments, the inhibitor of SOX21 binds to a SOX21 target site in the TGFβ2 nucleic acid sequence. This has the effect of blocking said target site (for example, by steric interference), preventing its recognition and binding by SOX21, and thus inhibiting SOX21 and its actions. In some embodiments, the SOX21 target site is located on the intron 1 of TGFβ2.

In a particular embodiment, the intron 1 of TGFβ2 has at least one of the regions comprising the following nucleic acid sequence: SEQ ID NO:3; SEQ ID NO:4; SEQ ID NO:5; SEQ ID NO:6; SEQ ID NO:13; SEQ ID NO:14; SEQ ID NO:15; SEQ ID NO:16 (mouse) and SEQ ID NO:7; SEQ ID NO:8; SEQ ID NO:9; SEQ ID NO:10; SEQ ID NO:11; SEQ ID NO:12; SEQ ID NO:17; SEQ ID NO:18; SEQ ID NO:19; SEQ ID NO:20; SEQ ID NO:21; SEQ ID NO:22 (human) as described below:

TABLE A
TGF/32 intron 1 sequences in mouse and human

| | Sense Sequences | Complementary sequences |
|---|---|---|
| Name | (= strand +) | (= strand−) |
| Mouse | SEQ ID NO: 3 | SEQ ID NO: 13 |
| Tgfβ2 intron 1 | 5′-AATTCATTGTTCTCTGG-3′ | 5′-CCAGAGAACAATGAATT- |
| sequence | | 3′ |
| Mouse | SEQ ID NO: 4 | SEQ ID NO: 14 |
| Tgfβ2 intron 1 | 5′-TGTTATTAAATTTAAA-3′ | 5′-TTTAAATTTAATAACA-3′ |
| sequence | | |
| Mouse | SEQ ID NO: 5 | SEQ ID NO: 15 |
| Tgfβ2 intron 1 | 5′-TAAATTTAAAATAAGT-3′ | 5′-ACTTATTTTAAATTTA-3′ |
| sequence | | |
| Mouse | SEQ ID NO: 6 | SEQ ID NO: 16 |
| Tgfβ2 intron 1 | 5′-TTTAAATTTAATAACA-3′ | 5′-TGTTATTAAATTTAAA-3′ |
| sequence | | |
| Human | SEQ ID NO: 7 | SEQ ID NO: 17 |
| Tgfβ2 intron | 5′-AACAATGATAGTTTT-3′ | 5′-AAAACTATCATTGTT-3′ |
| 1 sequence | | |
| Human | SEQ ID NO: 8 | SEQ ID NO: 18 |
| TGFβ2 intron | 5′-TTTCATTTTAAAAAAT-3′ | 5′-ATTTTTTAAAATGAAA-3′ |
| 1 sequence | | |
| Human | SEQ ID NO: 9 | SEQ ID NO: 19 |
| TGFβ2 intron | 5′-GCTGTTTTGTTTCTTTT-3′ | 5′-AAAAGAAACAAAACAGC- |
| 1 sequence | | 3′ |
| Human | SEQ ID NO: 10 | SEQ ID NO: 20 |
| TGFβ2 intron | 5′-GGGCATTTGTTTATCTC-3′ | 5′-GAGATAAACAAATGCCC- |
| 1 sequence | | 3′ |
| Human | SEQ ID NO: 11 | SEQ ID NO: 21 |
| TGFβ2 intron | 5′-TGTCATTTGTTTGGAAT-3′ | 5′-ATTCCAAACAAATGACA- |
| 1 sequence | | 3′ |
| Human | SEQ ID NO: 12 | SEQ ID NO: 22 |
| TGFβ2 intron | 5′-TTTAATAATATGTTAG-3′ | 5′-CTAACATATTATTAAA-3′ |
| 1 sequence | | |

In a further embodiment, the inhibitor of SOX21 inhibits SOX21 binding on the following nucleic acid sequences: SEQ ID NO: 3; SEQ ID NO: 4; SEQ ID NO: 5; SEQ ID NO:6; SEQ ID NO:7; SEQ ID NO: 8; SEQ ID NO: 9; SEQ ID NO:10; SEQ ID NO:11; SEQ ID NO:12.

In another embodiment, the inhibitor of SOX21 inhibits SOX21 binding on the following nucleic acid sequences: SEQ ID NO: 13; SEQ ID NO: 14; SEQ ID NO: 15; SEQ ID NO:16; SEQ ID NO:17; SEQ ID NO: 18; SEQ ID NO: 19; SEQ ID NO:20; SEQ ID NO:21; SEQ ID NO:22.

In some embodiments, the SOX21 inhibitor is an oligonucleotide wherein binding of said oligonucleotide to a SOX21 target site in the TGF (32 nucleic acid sequence may occur via complementary base pairing. Such oligonucleotide is complementary to a nucleic acid sequence of target site in the TGFβ2 (both sense and complementary sequences of TGFβ2 intron 1).

Thus, in some embodiments, binding between the SOX21 inhibitor oligonucleotide and the SOX21 target site in the TGFβ2 nucleic acid sequence occurs via complementary base pairing between at least one nucleotide present in the SOX21 inhibitor oligonucleotide and a corresponding nucleotide present in the SOX21 target site in the TGFb2 nucleic acid sequence, such that at least a portion of the SOX21 inhibitor oligonucleotide and the SOX21 target site in the TGF β2 nucleic acid sequence together define a base-paired nucleic acid duplex. Said complementary base pairing (and thus duplex formation) can occur over a region of two or more contiguous nucleotides of the SOX21 target site in the TGF β2 nucleic acid sequence (e.g. 3, 4, 5, 6, 7, 8, 9, 10, 11, 12, 13, 14, 15, 16, 17, 18, 19, 20 or 21 contiguous nucleotides). A base-paired nucleic acid duplex formed when the SOX21 inhibitor oligonucleotide binds to the SOX21 target in the TGFβ2 nucleic acid sequence (as described above) may comprise one or more mismatch pairings.

In some embodiments, two or more regions of complementary base-paired nucleic acid duplex (e.g. 3, 4, 5 or 6) are formed, wherein each region is separated from the next by one or more mismatch pairings.

In some embodiments, the target site is located on the intron 1 of TGFβ2. In some embodiments, wherein the SOX21 inhibitor oligonucleotide competes with SOX21 for binding to a SOX21 target site in the TGFβ2 nucleic acid sequence, the nucleic acid of said oligonucleotide comprises or consists of a nucleic acid sequence selected from the following sequences consisting SEQ ID NO: 3; SEQ ID NO: 4; SEQ ID NO: 5; SEQ ID NO:6; SEQ ID NO:7; SEQ ID NO: 8; SEQ ID NO: 9; SEQ ID NO:10; SEQ ID NO:11; SEQ ID NO:12; SEQ ID NO: 13; SEQ ID NO: 14; SEQ ID NO: 15; SEQ ID NO:16; SEQ ID NO:17; SEQ ID NO: 18; SEQ ID NO: 19; SEQ ID NO:20; SEQ ID NO:21; SEQ ID NO:22.

Thus, said nucleic acid sequence binds to the SOX21 target site located in the intron 1 of TGFβ2 via complementary binding at the location targeted by the seed region of SOX21, thus preventing SOX21 from binding.

Typically, such inhibitor blocks the interaction of SOX21 with TGFβ2 and thus decreases the activation of TGFβ2. In a particular embodiment, the inhibitor of SOX21 expression is a decoy. In the context of the invention, the decoy refers to a fragment of nucleic acid sequence or a variant of SOX21, such decoy is also called as transcription factor decoy.

In a particular embodiment, the transcription factor decoy is an oligodeoxynucleotide (ODN). In the context of the invention, the nucleic acid sequence (DNA or RNA) as described above binds to SOX21 which is a transcription factor. ODNs bearing the consensus binding sequence of SOX21. This strategy involves the intracellular delivery of such “decoy” ODNs, which are then recognized and bound by the target SOX21. Occupation of the transcription factor's DNA-binding site by the decoy renders the protein incapable of subsequently binding to the promoter regions of target gene such as TGFβ2. Such strategy is well-known in the art and described in J. Mann et al 2000: Therapeutic applications of transcription factor decoy oligonucleotides; J Clin Invest. 2000; 106(9):1071-1075.

The term “variant” is, with respect to nucleic acids, to be understood as a polynucleotide which differs in comparison to the nucleic acid from which it is derived by one or more changes in the nucleotide sequence. The nucleic acid from which variant is derived is also known as the parent nucleic acid. Typically, a variant is constructed artificially, preferably by gene-technological means. Typically, the parent nucleic acid is a wild-type nucleic acid or part thereof. The variants usable in the present invention may also be derived from homologs, orthologs, or paralogs of the parent nucleic acid. The changes in the nucleotide sequence may be exchanges, insertions, deletions, 5′ truncations, or 3′ truncations, or any combination of these changes, which may occur at one or several sites. In particular embodiment, a variant usable in the present invention exhibits a total number of up to 600 (up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500 or 600) changes in the nucleotide sequence. The nucleotide exchanges may be lead to non-conservative and/or preferably conservative amino acid exchanges as set out below with respect to polypeptide variants. Alternatively or additionally, a “variant” as used herein can be characterized by a certain degree of sequence identity to the parent nucleic acid from which it is derived. More precisely, a nucleic acid variant in the context of the present invention exhibits at least 80% sequence identity to its parent nucleic acid.

Particularly, the sequence identity of nucleic acid variants is over a continuous stretch of 20, 30, 40, 45, 50, 60, 70, 80, 90, 100, 150, 200, 250, 300, 400, 500, 600 or more amino acids, more preferably over the entire length of the reference nucleic acid (the parent nucleic acid). The term “at least 80% sequence identity” is used throughout the specification also with regard to nucleic acid sequence comparisons. This term preferably refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference nucleic acid or to the respective reference nucleic acid. Particularly, the nucleic acid in question and the reference nucleic acid or exhibit the indicated sequence identity over a continuous stretch as specified above.

In a further embodiment, the nucleic acid is an antisense oligonucleotide (AON) which is complementary to a nucleic acid sequence.

As used herein, the term “complementary” includes “fully complementary” and “substantially complementary”, meaning there will usually be a degree of complementarity between the oligonucleotide and its corresponding target sequence of more than 80%, preferably more than 85%, still more preferably more than 90%, most preferably more than 95%. For example, for an oligonucleotide of 20 nucleotides in length with one mismatch between its sequence and its target sequence, the degree of complementarity is 95%.

In particular, the invention relates to an antisense oligonucleotide complementary to a nucleic acid sequence of TGFβ2 (intron 1) that is necessary for the interaction with SOX21 having at least 25% sequence identity with target sequence.

According to the invention, a first amino acid sequence having at least 25% of identity with a second amino acid sequence means that the first amino acid sequence has 25%; 26%; 27%; 28%; 29%; 30%; 31%; 32%; 33%; 34%; 35%; 36%; 37%; 38%; 39%; 40%; 41%; 42%; 43%; 44%; 45%; 46%; 47%; 48%; 49%; 50%; 51%; 52%; 53%; 54%; 55%; 56%; 57%; 58%; 59%; 60%; 61%; 62%; 63%; 64%; 65%; 66%; 67%; 68%; 69%; 70%; 71%; 72%; 73%; 74%; 75%; 76%; 77%; 78%; 79%; 80%; 81%; 82%; 83%; 84%; 85%; 86%; 87%; 88%; 89%; 90%; 91%; 92%; 93%; 94%; 95%; 96%; 97%; 98%; 99% or 100% of identity with the second amino acid sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math, 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

Typically, said antisense oligonucleotides have a length of at least 15 nucleotides.

In a particular embodiment, the antisense oligonucleotide for the use according to the invention, wherein, said antisense oligonucleotide has a length of at least 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107 or 108 nucleotides.

In some embodiments, the inhibitor of SOX21 expression is a ribozyme. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of SOX21 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

In some embodiments, the inhibitor of SOX21 expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the error prone nonhomologous end-joining (NHEJ) and the high-fidelity homology-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisella 1 (Cpf1) in (Zetsche et al., 2015).

In another embodiment, the inhibitor of SOX21 activity is a peptide, polypeptide, peptidomimetic, small organic molecule, antibody or aptamers.

The term “polypeptide” refers both short peptides with a length of at least two amino acid residues and at most 10 amino acid residues, oligopeptides (11-100 amino acid residues), and longer peptides (the usual interpretation of “polypeptide”, i.e. more than 100 amino acid residues in length) as well as proteins (the functional entity comprising at least one peptide, oligopeptide, or polypeptide which may be chemically modified by being glycosylated, by being lipidated, or by comprising prosthetic groups).

In a particular embodiment, the polypeptide is a decoy peptide, polypeptide or peptidomimetic that is capable of binding to the intron 1 of TGFβ2.

In a particular embodiment, the peptidomimetic is a small protein-like chain designed to mimic a peptide such as SOX21 in the context of the invention. The term “peptidomimetic” or PM as used herein means a non-peptide chemical moiety. Peptides are short chains of amino acid monomers linked by peptide (amide) bonds, the covalent chemical bonds formed when the carboxyl group of one amino acid reacts with the amino group of another. The shortest peptides are dipeptides, consisting of 2 amino acids joined by a single peptide bond, followed by tripeptides, tetrapeptides, etc. A peptidomimetic chemical moiety includes non-amino acid chemical moieties. A peptidomimetic chemical moiety may also include one or more amino acid that are separated by one or more non-amino acid chemical units. A peptidomimetic chemical moiety does not contain in any portion of its chemical structure two or more adjacent amino acids that are linked by peptide bonds. The term “amino acid” as used herein means glycine, alanine, valine, leucine, isoleucine, phenylalanine, proline, serine, threonine, tyrosine, cysteine, methionine, lysine, arginine, histidine, tryptophan, aspartic acid, glutamic acid, asparagine, glutamine or citrulline.

In a particular embodiment, the peptidomimetic is a functional equivalent fragment of SOX21.

As used herein, a “functional equivalent” also known as a decoy, as “sink” or “trap” is a compound which is capable of binding to TGFβ2, thereby preventing its interaction with SOX21. Such peptidomimectic of SOX21 is in inactivated form. As used herein, a “functional equivalent” also known as a decoy or “decoy receptor”, as “sink” or “trap” is a compound which is capable of binding to TGFβ2 thereby preventing its interaction with SOX21. More particularly, it is a compound that binds to a ligand, but is structurally incapable of signalling or presenting the agonist to signalling receptor complexes. A decoy acts as a molecular trap for the ligand, thereby preventing it from binding to its functional receptor. A decoy can be a SOX21 peptidomimetic or a fragment thereof. The term “functionally equivalent fragment” thus includes any equivalent of SOX21 obtained by altering the amino acid sequence, for example by one or more amino acid deletions, substitutions or additions such that the protein analogue retains the ability to bind to soluble TGFβ2. Amino acid substitutions may be made, for example, by point mutation of the DNA encoding the amino acid sequence. Functional equivalents include molecules that bind TGFβ2.

The term “variant” is, with respect to peptidomimetics, to be understood as a peptidomimetic which differs in comparison to the peptidomimetic from which it is derived by one or more changes in the amino acid sequence. The peptidomimetic from which a protein variant is derived is also known as the parent polypeptide. Typically, a variant is constructed artificially, preferably by gene-technological means. Typically, the parent polypeptide is a wild-type protein or wild-type protein domain. The variants usable in the present invention may also be derived from homologs, orthologs, or paralogs of the parent polypeptide. The changes in the amino acid sequence may be amino acid exchanges, insertions, deletions, N-terminal truncations, or C-terminal truncations, or any combination of these changes, which may occur at one or several sites. In a particular embodiment, a variant usable in the invention exhibits a total number of up to 200 (up to 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, or 200) changes in the amino acid sequence (i.e. exchanges, insertions, deletions, N-terminal truncations, and/or C-terminal truncations). The amino acid exchanges may be conservative and/or non-conservative. In preferred embodiments, a variant usable in the present invention differs from the protein or domain from which it is derived by up to 1, 2, 3, 4, 5, 6, 7, 8, 9, 10, 15, 20, 25, 30, 35, 40, 45, 50, 55, 60, 65, 70, 75, 80, 85, 90, 95, or 100 amino acid exchanges, preferably conservative amino acid changes. Alternatively or additionally, a “variant” as used herein can be characterized by a certain degree of sequence identity to the parent polypeptide from which it is derived. More precisely, a protein variant in the context of the invention exhibits at least 80% sequence identity to its parent polypeptide. Particularly, the sequence identity of protein variants is over a continuous stretch of 20, 30, 40, 45, 50, 60, 70, 80, 90, 100, 150, 200, 250, 300, 400, 500, 600 or more amino acids, more preferably over the entire length of the reference polypeptide (the parent polypeptide). The term “at least 80% sequence identity” is used throughout the specification with regard to polypeptide sequence comparisons. This expression particularly refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide. Particularly, the polypeptide in question and the reference polypeptide exhibit the indicated sequence identity over a continuous stretch as specified above.

The sequence identity of a “variant” peptidomimetic of the invention can be determined over an identified range of an amino acid sequence of SOX21 or with reference to the entire amino acid sequence of an identified SOX21 decoy peptidomimetic. When determining the percent sequence identity for a variant peptidomimetic, the sequence alignment may omit any specifically excluded amino acid residues. For example, for a variant peptidomimetic of a SOX21 decoy peptidomimetic that exhibits at least 80% sequence identity to amino acids of SEQ ID NO: 2, the sequence alignment for comparison may occur across only amino acids or may take into account two or more identified ranges of amino acid sequences within the full length SOX21 decoy peptidomimetic.

In a particular embodiment, the inhibitor of SOX21 is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In some embodiments, the inhibitor of SOX21 is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see (Kabat et al., 1991), specifically incorporated herein by reference). Diabodies, in particular, are further described in EP 404, 097 and WO 93/11161; whereas linear antibodies are further described in (Zapata et al., 1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of (Beckman et al., 2007; Holliger & Hudson, 2005; Le Gall et al., 2004; Reff & Heard, 2001; Reiter et al., 1996; Young et al., 1995) further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388.

In a particular embodiment, the inhibitor of SOX21 is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the inhibitor is an intrabody having specificity for SOX21. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

In a particular embodiment, the inhibitor of SOX21 is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a further embodiment, the method according to the invention, wherein the inhibitor of SOX21 gene expression and/or activity as described above is delivered alone or in association with a viral vector.

Typically, the invention relates to a method of treating ocular disease in a subject in need thereof comprising a step of administering to said subject a therapeutically effective amount of a vector which comprises an inhibitor of SOX21 gene expression and/or activity.

In a particular embodiment, the method according to the invention, wherein the vector comprises a nucleic acid molecule as described above.

In a particular embodiment the nucleic acid molecule comprised in a vector encodes for an acid nucleic (such as siRNA, shRNA, miRNA, antisense oligonucleotide, ribozyme, or an endonuclease) specific to SOX21.

In another embodiment the nucleic acid molecule comprised in a vector encodes for an antisense oligonucleotide specific to the intron 1 of TGFβ2.

In a particular embodiment, the method according to the invention wherein the nucleic acid molecule is operatively linked to a promoter sequence (such as myocilin or dct promoters).

In a particular embodiment, the method according to the invention, wherein the vector is a viral vector.

In a particular embodiment, the method according to the invention, wherein the viral vector is lentivirus (LV).

As used herein, the term “lentivirus” refers to enveloped RNA particles measuring approximately 120 nm in size are efficient drug delivery tools and more particularly gene delivery tools. The LV binds to, and enters into target cells through its envelope proteins which confer its pseudotype. Once the LV has entered into the cells, it releases its capsid components and undergoes reverse transcription of the lentiviral RNA before integrating the proviral DNA into the genome of target cells. Non-integrative lentiviral vectors have been generated by modifying the properties of the vector integration machinery and can be used for transient gene expression. Virus-like particles lacking a provirus have also been generated and can be used to deliver proteins or messenger RNA. LV can be used for example, for gene addition, RNA interference, exon skipping or gene editing. All of these approaches can be facilitated by tissue or cell targeting of the LV via its pseudotype.

Lentivirus-like particles are described for example in (Aoki et al., 2011; Kaczmarczyk et al., 2011; McBurney et al., 2006; Muratori et al., 2010). Examples of lentivirus-like particles are VLPs generated by co-expressing in producer cells, a syncytin protein with a gag fusion protein (Gag fused with the gene of interest). The drug and/or syncytin may be, either displayed on the surface of the particles, or enclosed (packaged) into the particles. The syncytin protein is advantageously displayed on the surface of the particles, such as coupled to the particles or incorporated into the envelope of (enveloped) virus particles or virus-like particles to form pseudotyped enveloped virus particles or virus-like particles. The drug is coupled to the particles or packaged into the particles. For example, the drug is coupled to viral capsids or packaged into viral capsids, wherein said viral capsids may further comprise an envelope, preferably pseudotyped with syncytin. In some preferred embodiments, the drug is packaged into the particles pseudotyped with syncytin protein. The drug which is packaged into particles is advantageously a heterologous gene of interest which is packaged into viral vector particles, preferably retroviral vector particles, more preferably lentiviral vector particles.

In a particular embodiment, the method according to the invention, wherein the viral vector is adenovirus.

As used herein, the term “adenovirus” refers to medium-sized (90-100 nm), nonenveloped (without an outer lipid bilayer) viruses with an icosahedral nucleocapsid containing a double stranded DNA genome.

In a particular embodiment, the method according to the invention, wherein the viral vector is an adeno-associated virus (AAV) vector.

As used herein the term “AAV” has its general meaning in the art and is an abbreviation for adeno-associated virus, and may be used to refer to the virus itself or derivatives thereof.

The term covers all serotypes and variants both naturally occurring and engineered forms. According to the invention the term “AAV” refers to AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), and AAV type 8 (AAV-8) and AAV type 9 (AAV9). The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_001401 (AAV-2), AF043303 (AAV-2), and NC 006152 (AAV-5). As used herein, a “rAAV vector” refers to an AAV vector comprising the polynucleotide of interest (i.e. the polynucleotide encoding for the SOX21 polypeptide). The rAAV vectors contain 5′ and 3′ adeno-associated virus inverted terminal repeats (ITRs), and the polynucleotide of interest operatively linked to sequences, which regulate its expression in a target cell.

The AAV vector of the present invention typically comprises regulatory sequences allowing expression and, secretion of the encoded molecule polypeptide (i.e. the SOX21, peptidomimmetic), such as e.g., a promoter, enhancer, polyadenylation signal, internal ribosome entry sites (IRES), sequences encoding protein transduction domains (PTD), and the like. In this regard, the vector comprises a promoter region, operably linked to the polynucleotide of interest, to cause or improve expression of the protein in infected cells. Such a promoter may be ubiquitous, tissue-specific, strong, weak, regulated, chimeric, inducible, etc., to allow efficient and suitable production of the protein in the infected tissue. The promoter may be homologous to the encoded protein, or heterologous, including cellular, viral, fungal, plant or synthetic promoters. Examples of such regulated promoters include, without limitation, Tet on/off element-containing promoters, rapamycin-inducible promoters and metallothionein promoters. Examples of ubiquitous promoters include viral promoters, particularly the CMV promoter, CAG promoter (chicken beta actin promoter with CMV enhancer), the RSV promoter, the SV40 promoter, etc. and cellular promoters such as the PGK (phosphoglycerate kinase) promoter. The promoters may also be neurospecific promoters such as the Synapsin or the NSE (Neuron Specific Enolase) promoters (or NRSE (Neuron restrictive silencer element) sequences placed upstream from the ubiquitous PGK promoter), or promoters specific for iris cell types such as DCT or the trabeculum meshwork such as MYOC, or retinal cell types such as the RPE65, the BEST1, the Rhodopsin or the cone arrestin promoters. The vector may also comprise target sequences for miRNAs achieving suppression of transgene expression in non-desired cells. In some embodiments, the vector comprises a leader sequence allowing secretion of the encoded protein. Fusion of the polynucleotide of interest with a sequence encoding a secretion signal peptide (usually located at the N-terminal end of secreted polypeptides) will allow the production of the therapeutic protein in a form that can be secreted from the transduced cells. Examples of such signal peptides include the albumin, the β-glucuronidase, the alkaline protease or the fibronectin secretory signal peptides.

The recombinant AAV vector of the present invention is produced using methods well known in the art. In short, the methods generally involve (a) the introduction of the rAAV vector into a host cell, (b) the introduction of an AAV helper construct into the host cell, wherein the helper construct comprises the viral functions missing from the rAAV vector and (c) introducing a helper virus into the host cell. All functions for rAAV virion replication and packaging need to be present, to achieve replication and packaging of the rAAV vector into rAAV virions. The introduction into the host cell can be carried out using standard virological techniques simultaneously or sequentially. Finally, the host cells are cultured to produce rAAV virions and are purified using standard techniques such as CsCl gradients. Residual helper virus activity can be inactivated using known methods, such as for example heat inactivation. The purified rAAV vector is then ready for use in the method of the present invention.

In a particular embodiment, the method according to the invention, wherein the AAV vector is selected from vectors derived from AAV serotypes having tropism for and high transduction efficiencies in ocular cells.

In a particular embodiment, the method according to the invention, wherein the AAV vector is an AAV1, AAV2, AAV3, AAV4, AAV 5, AAV 6, AAV7, AAV 8 or AAV9.

In a particular embodiment, the method according to the invention, wherein the AAV vector is an AAV1, AAV 2, AAV 5, AAV 7, 8 or AAV 9.

As used herein the terms “administering” or “administration” refer to the act of injecting or otherwise physically delivering a substance as it exists outside the body (e.g., an inhibitor of SOX21 naked or with a viral vector) into the subject, intravenous, intravitreal, subcutaneous administration (e.g., by injection or infusion). When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

Administering the recombinant AAV vector of the present invention to the subject is preferably performed by intravenous, intravitreal, subcutaneous delivery. In some embodiments, the recombinant AAV vector of the present invention is administered to the subject by the intravitreous injection.

In a particular embodiment, the method according to the invention, the inhibitor of SOX21 naked or with a viral vector according to the invention is delivered by intravitreous, subcutaneous, intravenous, ophthalmic drop or ophthalmic ointment delivery.

In another embodiment, the inhibitor of SOX21 naked or with a viral vector according to the invention is delivered for an ophthalmic drop or an ophthalmic ointment use.

In a further embodiment, the inhibitor of SOX21 naked or with a viral vector according to the invention is delivered by electroporation or sonoporation.

In a particular embodiment, the inhibitor of SOX21 naked or with a viral vector according to the invention is delivered to the iris, the ciliary body, aqueous humor or the trabeculum meshwork.

By a “therapeutically effective amount” of inhibitor of SOX21 gene expression and/or activity alone or in association with a viral vector (e.g. AAV) as above described is meant a sufficient amount of the inhibitor alone or inhibitor with a viral vector for the treatment of ocular disease (glaucoma, OAG, POAG, myopia). It will be understood, however, that the total dosage of the AAV vector of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. Typically, from 10⁸ to 10¹⁰ viral genomes (vg) are administered per dose in mice. Typically, the doses of AAV vectors to be administered in humans may range from 10¹⁰ to 10¹² vg.

### Pharmaceutical Composition

The inhibitor of SOX21 expression and/or activity (alone or with a vector) as described above may be combined with pharmaceutically acceptable excipients, and optionally sustained-release matrices, such as biodegradable polymers, to form pharmaceutical compositions.

Accordingly, the invention relates to a pharmaceutical composition comprising an inhibitor of SOX21 expression and/or activity.

In a particular embodiment, the pharmaceutical composition comprising an inhibitor of SOX21 expression and/or activity alone or in association with a viral vector.

In a particular embodiment, the pharmaceutical composition according to the invention for use in the treatment of ocular disease.

More particularly the pharmaceutical composition according to the invention for use in the treatment of MCOR.

In a further embodiment, the pharmaceutical composition according to the invention for use in the treatment of glaucoma.

In a particular embodiment, the pharmaceutical composition according to the invention for use in the treatment of OAG including POAG.

In a particular embodiment, the pharmaceutical composition according to the invention for use in the treatment of myopia.

As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for intravenous, intravitreal or subcutaneous delivery, the active principle, alone or in combination with another active principle, can be administered in a unit administration form, as a mixture with conventional pharmaceutical supports, to animals and human beings. Suitable unit administration forms comprise intravenous, intravitreal, and subcutaneous administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In a particular embodiment, the inhibitor of SOX21 according to the invention (naked or with vectors of the invention) may be delivered in a pharmaceutically acceptable ophthalmic vehicle, such that the inhibitor (naked or with vectors of the invention) can penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically-acceptable ophthalmic vehicle may, for example, be an ointment, vegetable oil or an encapsulating material.

Alternatively, the inhibitor of SOX21 according to the invention (naked or with vectors of the invention) may be injected directly into the vitreous, aqueous humour, iris, ciliary body tissue(s) or cells and/or extra-ocular muscles, retina (e.g. after retinal detachment) or even in the suprachoridal space. Electroporation or sonoporation means may also be suitable for delivering the inhibitor of SOX21 according to the invention (alone or with vectors of the invention). In a further embodiment, the inhibitor of SOX21 naked or with a viral vector is formulated in a pharmaceutically acceptable ophthalmic vehicle for an ophthalmic drop or an ophthalmic ointment use.

### Method of Screening

A further object of the present invention relates to a method of screening a drug suitable for the treatment of ocular disease (glaucoma, OAG, POAG and/or myopia) comprising i) providing a test compound and ii) determining the ability of said test compound to inhibit the expression and/or activity of SOX21.

Any biological assay well known in the art could be suitable for determining the ability of the test compound to inhibit the expression and/or activity of SOX21.

In some embodiments, the assay first comprises determining the ability of the test compound to bind to the SOX21 gene, its mRNA or its product. In some embodiments, the assay first comprises determining the ability of the test compound to bind to the intron 1 of TGFβ2. In some embodiments, a population of cells is then contacted and activated so as to determine the ability of the test compound to inhibit the expression and/or activity of SOX21. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the expression and/or activity of SOX21, as determined using in vitro methods described herein, are likely to exhibit similar modulatory capacity in applications in vivo. Typically, the test compound is selected from the group consisting of peptides, petptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form nucleic acids such as antisense.

The invention will be further illustrated by the following figures and examples. However, these examples and figures should not be interpreted in any way as limiting the scope of the present invention.

### FIGURES

FIG. 1. Pupillary response, expression level analysis at the 1 Mb-TAD, RTqPCR, WB and HIC analysis of Sox21 in cΔMCOR and WT animals. (A) Pupil diameter as determined by pupilometry show moderate, yet statistically significant basal reduction of the pupil size in cΔMCOR as compared toWT animals (**: p<0.01, n=8 animals, each group); Pupil size upon mydriactic administration (neosynephrin, 10 min) was similar in the two mouse lines (ns: not significant). (B) Abundance of genes as determined by RNAseq. Note that RNAseq abundance is represented by the log [Desq Normalized counts] to allow the representation of all genes which display highly variable levels of expression. The abundance of Dzip1, Dnajc3 and Uggt2 differs in cΔMCOR and WT but the fold of differential expression is <1.5 at p<0.05 (RNAseq analysis cutoff). Consistent with low difference among cΔMCOR and WT samples, semi-quantitative RTqPCR analysis failed to show deregulation. (C) RTqPCR analysis of Dct and Sox21 abundance in iris/ciliary body RNA extracts form newborn cΔMCOR and WT mice (n=5, each group).

FIG. 2. TGFβ2 genomic sequences binding SOX21. (A) Mouse sequence identified by CHIP-seq that binds SOX21 in the iris of cΔMCOR mice and (B) Human synthetic sequence. The underlined sequences correspond to the murine (SOX21.1; blue) and human (SOX21; green) consensus SOX21-binding sites.

FIG. 3. Analysis of TGFB2 concentration in the aqueous humor and preliminary analysis of optic nerve head integrity in cΔMCOR and WT mice. ELISA dosage of TGFB2 in the aqueous of (A) 12-month-old mice (9 mice for each genotype) and (B) in human samples showing accumulation in the cΔMCOR mice (**p<0.01) and in one MCOR patient. (C) HE staining of optic nerve heads of cΔMCOR and WT mice (n=1 each genotype). Glial cells can be seen by a marked coloration. Glial cell counts show that their abundance is highly decreased in cΔMCOR mice (****p<0.0001).

FIG. 4. Immunocytochemistry analysis of non-edited (A, B, C) and edited cells (D, E, F) SV40-hIPEpiC cells. DCT is seen in the cytoplasm of non-edited and edited cells (A, D). SOX21 is seen in some nuclei of edited cells (D, E) but not non-edited cells. C and F show nuclei stained with DAPI. Scale bar, 10 um.

FIG. 5. SOX21 abundance in RPE1, MP41 and OCM-1 relative to glioma cells, as determined by real time RT-PCR. Data shows no induction of SOX21 expression in edited cells carrying the critical MCOR deletion in heterozygosity (HT) compared to non-edited (WT) cells.

### EXAMPLE 1

Material & Methods

Mouse Lines Transgenics mice were generated by Imagine Transgenic Platform using a CRISPR/Cas9 system. All animal procedures were performed with approval from the Ministry of Higher Education, Research and Innovation and the ethical committee of the Paris Descartes University. Guide RNAs (sgRNAs, Table 1) were designed via the CRISPOR (http://crispor.tefor.net/) and sequences are listed in the table below. C57BL/6J female mice (4 weeks old) were superovulated by intraperitoneal injection of 5 IU PMSG (SYNCRO-PART® PMSG 600 UI, Ceva) followed by 5 IU hCG (Chorulon 1500 UI, Intervet) at an interval of 46 h-48 h and mated with C57BL/6J male mice. The next day, zygotes were collected from the oviducts and exposed to hyaluronidase (H3884, Sigma-Aldrich) to remove the cumulus cells and then placed in M2 medium (M7167, Sigma-Aldrich) into a CO2 incubator (5% CO2, 37° C.).SgRNAs were hybridized with cas9 (WT) protein and injected into the pronucleus of the C57Bl/6J zygotes. Surviving zygotes were placed in KSOM medium (MR-106-D, Merck-Millipore) and cultured overnight to two-cell stage and then transfered into the oviduct of B6CBAF1 pseudo-pregnant females. The generated transgenic mice were validated by Sanger sequencing combined with tide TIDE analysis (https://tide-calculator.nki.nl/; data not shown). All mice were backcrossed with C57BL/6j mice to remove potential off-targets. The offspring were further confirmed by PCR genotyping with appropriate primers.

TABLE 1
Guide RNA used to generate MCOR mice models

| Mouse lines | 5′ Guide (5′-3′) | 3′ Guide (5′-3′) |
|---|---|---|
| cΔMCOR | CTCACAGTTTGGT | ATTCCCCAGCAGAG |
| | CCAGGCTGGG | AGGCGCTGG |
| | (SEQ ID NO: 23) | (SEQ ID NO: 29) |
| ΔCTCF1 + Ps | TCTTCAGACGCCG | GCCCGCTCCGTTTG |
| | CGCTTTA | CTCGCC |
| | (SEQ ID NO: 24) | (SEQ ID NO: 30) |
| ΔCTCF2 | GTGTTTTATGGACG | CTCGGCATAAAGTT |
| | GGCTCG | TGTAAT |
| | (SEQ ID NO: 25) | (SEQ ID NO: 31) |
| ΔCTCF3 | TCCATAGTAATGAT | CTCGGCATAAAGTT |
| | CGCATC | TGTAAT |
| | (SEQ ID NO: 26) | (SEQ ID NO: 32) |
| ΔEnh1 | AACACAGGGAGGTC | CAAAAATCCTTGGG |
| | GCTTTC | CTAACT |
| | (SEQ ID NO: 27) | (SEQ ID NO: 33) |
| ΔEnh2 | TGGGACACAAGCAC | GTTCCAGTAGGGCA |
| | CGGCCT | ACGCAA |
| | (SEQ ID NO: 28) | (SEQ ID NO: 34) |

Circular Chromosome Conformation Capture Sequencing (4C-Seq).

Potential active enhancers and silencers specific to cΔMCOR mice were assessed for gain or loss of interaction with the Sox21 promoter by using 4C-seq technology. We performed 4C-seq from the viewpoint of 2 kb surrounding the Sox21 regulating region, in embryos as well as in mice embryonic fibroblast (MEF) derived from WT and cΔMCOR mice (E9.5). Briefly, genomic interactions were captured by cross-linking and chromatin aggregates which underwent two rounds of digestions (i.e. DpnII and Csp6I; New England Biolabs), ligation-induced circularization of short digested fragments, inverse PCR amplification using primers designed to target the viewpoint fragment (5′ tgctcccctgttatgttcagatc 3′ (SEQ ID NO: 35) and 5′gtgcaaaccaattcatgtta 3′(SEQ ID NO: 36) and amplification of its ligated partners as described by van de Werken et al., 2012 and Lupiáñiez et al., 2015. A total of 1.6 mg of each library was PCR amplified and barcoded to allow 50 bp single-end read sequencing with the Illumina Nova-seq technology. High resolution contact profiles in the 2 Mb region surrounding the viewpoint were generated by using 4Cseqpipe that allow sequence extraction, mapping, normalization, and plotting of cis-contact profiles around viewpoints (van de Werken et al. 2012). In short, 4C-seq reads were demultiplexed and cleaned of the primer sequences. Trimmed reads were mapped against the human genome assembly GRCh38 (Bowtie2 2.2.3 with default setting) and filtered-out for low mapping quality and nonunique sequences (mapping scores MAPQ <30; Samtools 0.1.19). To calculate read count profiles, the viewpoint and adjacent fragments 1.5 kb up- and downstream were removed. A sliding window of 10 Kb was used to smooth the data. Data was normalized to reads per million mapped reads (RPM) to account for depth of sequencing of the 4C-seq library by scaling all reads mapped to the chromosome containing the viewpoint. To compare interaction profiles of the different samples, the log 2 fold change for each window of normalized reads were calculated. To obtain ratios, duplicated regions were excluded for calculation of the scaling parameter used in RPM normalization. Two-way ANOVA with multiple comparisons was used to compare differential contacts between B6.WT and B6.cDMCOR in embryos as well as in MEF to obtain the adjusted Pvalue. Results for each group were calculated and assessed for statistical significance using the 2-tailed unpaired Student's t-test.

Pupillometry

Two-month-old mice (8 WT and 8 cΔMCOR) were dark-adapted overnight. Pupil diameter was recorded as previously described by Kostic et al 2016. In brief, the baseline pupil diameter was set as the mean pupil diameter during the 500 ms before light onset; thereafter, all pupil sizes were converted to a relative size that was a function of the baseline value. The following light stimulus sequence was used: 50 ms (−2.2, −1 and 0.5) log W/m² white light and 20 s 0 log W/m² blue light. The pupil diameter was determined automatically by the Neuroptics A2000, Inc. software. One-way/two-way ANOVA analysis were used to identify significant differences.

RNAseq

Total iris RNA from WT and cΔMCOR mice were extracted using the RNeasy Mini Kit (Qiagen). RNAseq was performed at the Genomic Platform of Imagine. In brief, total RNAs (200 ng) were purified, fragmented, reverse transcribed and barcoded. cDNA libraries were prepared from 4 WT and 4 cΔMCOR samples using the TruSeq RNA Sample Preparation Kit, according to the manufacturer recommendations (Illumina). Indexed cDNA libraries were pooled and hybridized to biotin-labeled probes specific for coding RNA regions. Bound cDNAs were recovered using streptavidin-bead mediated purification and hybridized for a second enrichment reaction, prior to clonal amplification by cluster generation and sequencing on a HiSeq 2000 (Illumina). Analysis of RNA-seq data were performed at the Bioinfoimatics Platform of Imagine using a standard workflow including quality assessment (FastQC 0.11.5), quality filtering (Trimmomatic), read mapping against the human genome assembly GRCh38 (STAR aligner), read counting (HTSeq software with annotation from GENCODE v24 http://www.gencodegenes.org/). Gene expression levels were normalized and compared among samples using LimmaVoom, DESeq2 and edgeR. Mean expression values of genes displaying at least a 1.5-fold change (p<0.05) in cΔMCOR group compared to WT group were analyzed for hierarchical and functional clustering using the Partek Genomics Suite 6.6 that includes ANOVA and Ingenuity Pathway Analysis (http://www.ingenuity.com) modules.

RT-qPCR Analysis

Adult mice were sacrificed by cervical dislocation and enucleated. The eyes were carefully dissected to recover the iris and CB. Iris total RNA (200 ng) from WT and cΔMCOR mice was extracted using the RNeasy Mini Kit (Qiagen) and subjected to reverse transcription with the Reverse Transcriptor kit following the manufacturer's instructions (Roche). The abundance of Sox21 and Dct mRNA were measured using specific primers: Sox21 (5′-gatgcacaactcggagatca-3′(SEQ ID NO: 37)/5′-ggcgaacttgtcctttttga-3′(SEQ ID NO: 38) and Dct (5′-aattcttcaaccggacatgc-3′(SEQ ID NO: 39)/5′-ttgcgtggtgatcacgtagt-3′(SEQ ID NO: 40). GusB (5′-ctgcggttgtgatgtggtctgt-3′(SEQ ID NO: 41)/5′-tgtgggtgatcagcgtcttaaagt-3′(SEQ ID NO: 42) and Hprt 1 (5′-gttggatacaggccagactttgtt-3′(SEQ ID NO: 43)/5′-aaacgtgattcaaatccctgaagta-3′(SEQ ID NO: 44) were used to normalize the data and Alb (5-gggacagtgagtacccagacatcta-3′(SEQ ID NO: 45)/5′-ccagacttggtgttggatgctt-3′(SEQ ID NO: 46) was used to control the non-contamination of cDNAs by genomic DNA. The cDNA (5 μl of a solution diluted at 1:25 in RNAse-free H₂O) of each sample was subjected to PCR amplification in real-time in a buffer (200 containing SYBR GREEN PCR Master Mix (Life Technologies) and 300 nM forward and reverse primers in the following conditions: activation of Taq polymerase and denaturation at 95° C. for 10 min followed by 50 cycles of 15 s at 95° C., and 1 min at 60° C. The specificity of the amplified products was determined after the analysis of the melting curve carried out at the end of each amplification using one cycle at 95° C. for 15 s, then a graded thermal increase of 60° C. to 95° C. for 20 min. The data analysis and methodology were performed as previously described by Gerard et al 2012.

Immunoblotting

Adult mice were sacrificed by cervical dislocation and enucleated. The eyes were carefully dissected to recover the iris and CB. Tissues were lysed on ice for 1 h by repeated homogenization in a low detergent lysis buffer containing phosphate buffered saline (PBS) 1×, 1% Triton, Halt™ Protease Inhibitor Cocktail 1× (ThermoScientific) and 25 U/ml Pierce Universal Nuclease (ThermoScientific). The lysates were centrifuged (20 000 g at 4° C. for 15 min), supernatants were collected and proteins quantified using the Bradford method. For western blot analysis, proteins (25 μg) were resolved by a 4-15% polyacrylamide gel (mini-PROTEAN TGX, Bio-Rad, Marnes-la-Coquette, France) according to the supplier's recommendations. All lysates were heated at 95° C. for 10 min prior to loading. Proteins were transferred to a PVDF 0.2 μM membrane (Bio-Rad) using a Trans-Blot Turbo Transfer System (Bio-Rad), and then processed for immunoblotting. Membranes were probed with Sox21 goat polyclonal (1:2000, AF3538 R&D system) and monoclonal mouse anti-β-actin (1:2000, Abcam, Paris, France) primary antibodies, and then incubated with donkey anti-goat IgG-HRP (1:2000, ThermoScientific) and donkey anti-mouse IgG-HRP secondary antibodies (1:4000, ThermoScientific), respectively. Blots were developed with the use of the Clarity Western ECL Substrate (Bio-Rad) and ChemiDoc XRS+ Imaging System (Bio-Rad). Western blot images were acquired and analyzed with Image Lab software 3.0.1 build 18 (Bio-Rad).

Immunofluorescence Labeling

For immunohistochemistry C57BL/6J cΔMCOR mice were derived on an albino background which expresses Dct (Swiss-albino, CFW). Adult mice were sacrificed by cervical dislocation and enucleated. Eyes were dissected in phosphate buffered saline (PBS), fixed in 4% paraformaldehyde overnight at 4° C. and washed three times 15 min with 1×PBS. Eyes were embedded in OCT™ Compound and stored at −80° C. Sagittal sections (10 μm) were first soaked for 30 min in a buffer solution for heat induced epitope retrieval (10 mM sodium citrate, 0.05% Tween 20, pH6) then cooled for 30 min at room temperature and blocked for 1 hour with 5% BSA/PBS 1×. Overnight primary antibody incubation (SOX21 goat polyclonal, AF3538 R&D system 1:100 and DCT rabbit anti mouse, ab74073 Abcam 1:200) was performed at 4° C. followed by Alexa Fluor secondary antibody incubation for 1 hour at room temperature (Donkey anti-goat 647 A 21447 and Donkey ant rabbit 555 A31572 at 1:1000 dilution). All sections were counterstained with Dapi (Sigma10236276001) for visualization of nuclei. Images were taken using Spinning Disk (Zeiss) fluorescent microscope and images were analyzed using the image J analysis system.

Glial Nuclei Counting in the Optic Nerve Head.

One-year-old WT and cΔMCOR mice were sacrificed by cervical dislocation and enucleated. Optic nerves (ON) were dissected from globe by using curved scissors, fixed in 4% paraformaldehyde, embedded in paraffin and cut in 4 μm thick cross sections. Sections were stained using hematoxylin-eosin to label the nuclei of glial cells which were counted on multiple sections (n=35 and 38 for WT and cΔMCOR, respectively) using ImageJ software (Wayne Rasband, NIH, USA). The means of nuclei per slice were compared using a bilateral heterodastic Student test.

ELISA Dosage of TGFβ2 Concentration in the Aqueous Humor

Adult mice were sacrificed to allow aqueous humor (AH) collection (5 μl). Human AH (approximately 100 μl) was collected in the course of senile cataract surgery in one MCOR individual and 11 controls. Mouse TGFβ2 DuoSet ELISA kit (R&D Systems) and human TGFβ2 DuoSet ELISA kit (catalogue No DB250 R&D Systems) were utilized to quantify total TGFβ2 levels in mice and human AH (10 μl and 30 μl in 100 μl final, respectively). Samples were subjected to acid activation (1N HCl) and neutralization (1.2N NaOH/0.5 M HEPES) prior to quantification of the total TGFβ2 concentration as recommended by the manufacturer (R&D Systems), using a microplate reader at a 450-nm wavelength with a 570-nm wavelength correction (reference du lecteur de plaque). Statistical significance between two groups was analyzed using the unpaired 2-tailed Student's t test. p≤0.05 was considered statistically significant.

Results

Primary mapping of the disease locus to chromosome 13q32 has been achieved in Necker Hospital, Paris at the end of the 1990s (Rouillac et al., 1998) by whole genome-linkage analysis in the 5-generation Breton family ascertained in the 1960s. Genetic analysis in other MCOR families suggests that there exist only one MCOR locus (Fares-Taie et al., 2015; Pozza et al., 2020; Ramprasad et al., 2005; Sergouniotis et al., 2017). Consistently, studying the multigenerational Breton pedigree and five other families entrusted to the inventors, one French, two Mexican and two Japanese families, the inventors were able to ascribe all the cases to submicroscopic overlapping 13q32.1 deletions (Fares-Taie et al., 2015). The Mexican families shared the same deletions, suggesting a founder effect. All other families displayed unique anomalies. The deletions were variable in size (35-85 kb) but encompassed or interrupted invariably the tail-to-tail genes, TGDS and GPR180. Recessive mutations in TGDS encoding the TDP-glucose 4,6-dehydratase cause oro-facio-digital malformations (Catel-Manzke syndrome, CMS; MIM616145). Ophthalmological examination of individuals affected with TGDS-associated CMS from our hospital revealed absence of iris anomalies, suggesting a lack of role of this gene in MCOR. GPR180 encoding a G protein-coupled receptor 180 of unknown function is involved in the regulation of smooth muscle cell growth (Iida et al., 2003). However, studying knock-out mice and individuals from a two-generation family carrying a heterozygous GPR180 nonsense mutation, the inventors observed no iris dilator muscle anomaly (Fares-Taie et al., 2015). The family members harbouring the GRP180 loss-of-function mutation, aged from 16 to 62 years displayed some minor irido-corneal angle anomalies (iris spicules) with normal IOPs (Fares-Taie et al., 2015). Another 69 Kb overlapping deletion has been reported in a 3-generation family from UK comprising five affected individuals with irido-corneal angle anomalies in at least three aged over 40, two of whom (mother and son) had juvenile GLC (Sergouniotis et al., 2017). Recently, a reciprocal 289 kb duplication encompassing 11 genes including TGDS and GPR180 has been identified in a mosaic mother and her daughter with normal anterior chamber angle (Pozza et al., 2020). Together, these observations suggest that the loss of GPR180 might contribute to angle anomalies, but is insufficient to explain the disease that is likely due to alteration of the 13q32.1 regulatory landscape.

HiC sequencing data suggest that the MCOR locus is included in a 1 Mb topologically-associated domain (TAD) comprising region from Dct encoding the dopachrome tautomerase that acts downstream of the tyrosinase in the biosynthesis pathway of eumelanin, to Uggt2 encoding the UDP-glucose glycoprotein glucosyltransferase 2 that selectively reglucosylates unfolded glycoproteins in the endoplasmic reticulum (Bonev et al., 2017). The region and its 3D structure are highly conserved in the mouse syntenic region on chromosome 14qE4. Using the CRISPR/Cas9 methodology, the inventors generated transgenic mice harbouring the critical MCOR deletion (cΔ; 35 Kb) or smaller deletions within or outside the critical region (data not shown) in order to characterize its regulatory architecture and understand its relation to iris development, OAG and high myopia.

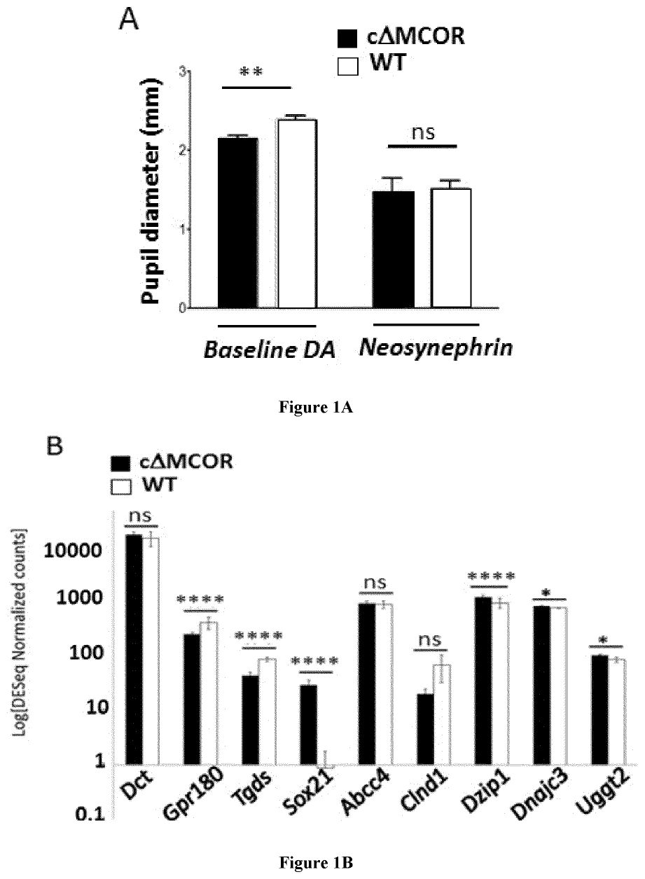

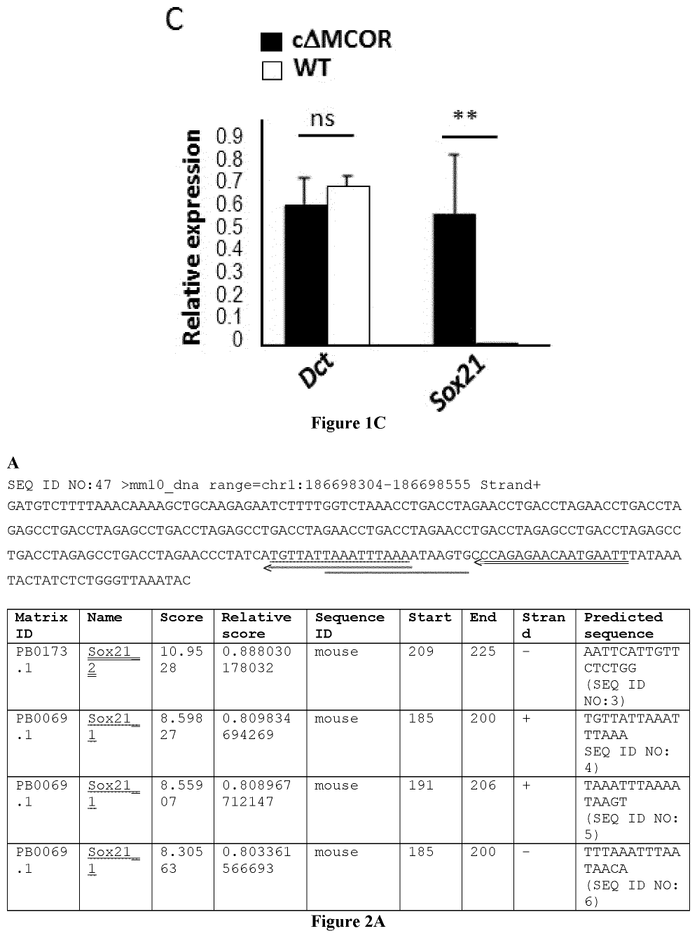

Studying the resulting phenotypes, the inventors observed that the loss of Tgds in homozygosity caused embryonic mortality at E9.5 due to neural crest migration and differentiation anomalies (at least in part). In contrast, mice carrying the critical deletion in heterozygosity (cΔMCOR mice) are viable and present with a moderate reduction in base-line pupil size compared to WT littermates (p<0.01) (FIG. 1A). Analysing the expression levels of genes flanking the cΔ within the TAD (FIG. 1B) from RNAseq datasets generated from new-born cΔMCOR and WT irises, the inventors observed an ectopic expression of SOX21. None of the other genes flanking the cΔ, were deregulated (FIG. 1B). SOX21 mRNA (RTqPCR) and its product (Western Blot) were detected in the iris of the cΔMCOR mouse (FIG. 1 and data not shown) starting from E16 through to adulthood (not shown), whereas they were undetectable in the WT.

SOX21 encodes a transcription factor of the SRY-related HMG-box (SOX) family, which only known function in the eye comes from studies in the chick and zebrafish (Lan et al., 2011; Uchikawa et al., 1999). In the chick, SOX21 is transiently activated during the early phases of optic vesicle morphogenesis and specification in the lens and retina but no longer expressed afterwards (Uchikawa et al., 1999). The ocular expression of SOX21 stops before the iris starts developing. Its loss-of-function in the chick, as in zebrafish, interferes with normal lens development (Pauls et al., 2012).

The CCCTC-containing protein also known as CTCF is a highly conserved zinc finger protein that binds chromatin and mediates its 3D organization through looping between binding sites. It can function as a transcriptional activator, a repressor or an insulator protein, blocking the communication between enhancers and promoters (Holwerda & de Laat, 2013). We identified 4 CTCF-binding sites within the 35 Kb critical MCOR region (data not shown). To assess whether the loss of one or several of them could promote the adoption by the promoter of SOX21 of a nearby active enhancer, the inventors ablated them individually or in combination in the mouse (data not shown). The iris of none of the resulting mouse lines displayed SOX21 expression, as determined by RTqPCR (data not shown). This observation supports the view that ectopic expression of SOX21 in the iris of the cΔMCOR mouse is not due to the loss of insulator. In addition, the inventors performed 4C sequencing (4Cseq) from a viewpoint of 2 kb surrounding the SOX21 promoter to investigate how the cΔMCOR influences chromatin interactions between SOX21 and active enhancers and search for new interactions. 4Cseq of nuclei isolated from WT and cΔMCOR total embryos and embryonic fibroblasts (E9.5) was in agreement with chromatin structure of the 1 Mb TAD reported at the locus (Bonev et al., 2017). Intriguingly, 4C-seq revealed that the DNA region encompassing SOX21 (2 kb) interacts throughout the TAD in both cΔMCOR and WT counterparts (data not shown) that do not express SOX21, suggesting either iris-specific interactions or a modification by the deletion of the SOX21 promotor competence for nearby enhancers. Using CHIP Seq for H3k27ac marks which indicates transcriptionally active chromatin sites (Raisner et al., 2018) in irises of newborn WT mice, the inventors identified two highly active enhancers upstream of the deletion, nearby Dct (data not shown) that is highly expressed in pigmented iris cells (data not shown). It is likely that the SOX21 promoter adopts one or the two enhancers by reducing genomic distances within the TAD by deletions or duplications.

Immunohistochemistry (IHC) analysis in cΔMCOR is hampered by the strong iris pigmentation. Depigmentation protocols did not allow preserving the integrity of iris tissues. Hence, the inventors derived the line on a tyrosinase (tyr)-negative albino background. RTqPCR analysis confirmed SOX21 ectopic expression in the iris of 2 month-old c.ΔMCOR mice (data not shown). IHC showed SOX21 expression in the iris PEL and CB the stained using an antibody specific to DCT (Dct is endogenously highly expressed in both cΔMCOR and WT) (data not shown). SOX21 was undetectable in the iris AEL that form the dilator muscle. This might suggest a remote effect of the aberrant gene expression in the iris PEL and CB on the iris AEL However, while pupilometry analysis showed reduced pupil size in cΔMCOR mice, preliminary IHC analysis have not permitted substantiating dilator muscle anomalies as there existed SMA-positive fibers in the dilator muscle region of cΔMCOR irises with no visible difference as compared to WT (not shown). The presence of SMA-positive fibers is not unexpected considering that the pupil size is only moderately reduced in the model.

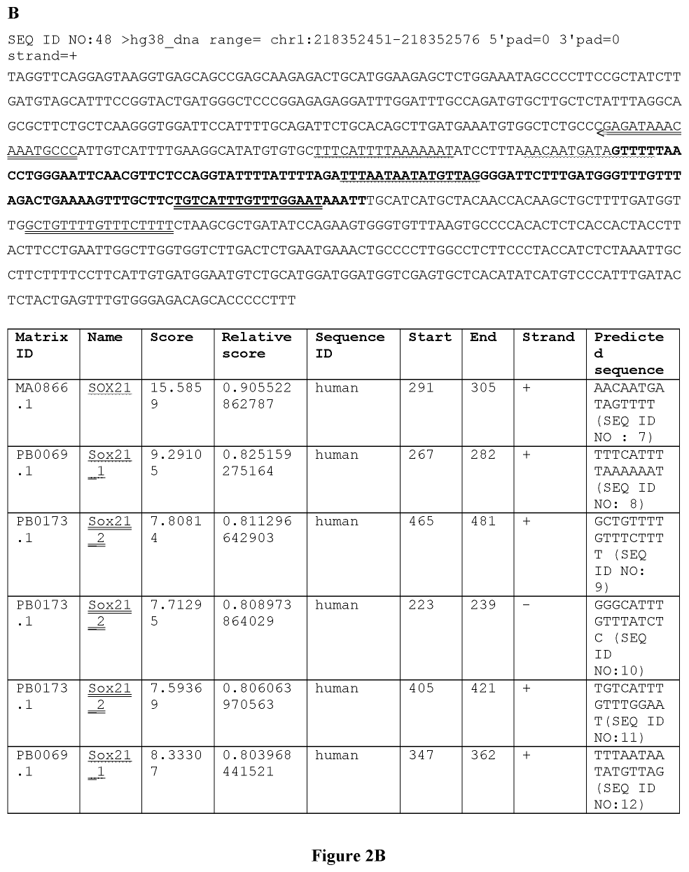

Analyzing data from RNAseq of irises from new-born cΔMCOR and WT, the inventors identified 2500 deregulated genes (≥1.5 fold, p<0.05) in the cΔMCOR model. Many of them are in relation with MCOR disease symptoms and/or iris development, e.g. Des (0.49, p=0.012) encoding desmin intermediate filaments which lacks in iris AEL of patients affected with MCOR, Wtn2b (2.35 p<0.01) which expression is pivotal for the specification of iris progenitor cells to a non-neuronal (myoepithelial) fate, Bmp7 (1.47 p<0.05) that is highly expressed by cells at the site of iris smooth muscle generation and Tgfβ2 and Gdnf (1.6 and 1.7 respectively, p<0.01) encoding two closely related growth factors involved in GLC (Checa-Casalengua et al., 2011; Kasetti et al., 2017; Prendes et al., 2013) and high myopia (Jia et al., 2017). Interestingly, CHIPseq analysis of irises from new-born.cΔMCOR mice using a highly specific SOX21 antibody (Matsuda et al., 2012) showed binding of SOX21 on 26 DNA regions through-out the genome. Getting back to RNAseq dataset from cΔMCOR and WT irises, the inventors observed that 2/26 binding regions were included in genes that were deregulated (>1.5 fold at a p<0.05) in .cΔMCOR: Tgfβ2 and Gdnf. The other 24 DNA regions lie in genes that were not deregulated (11/24) in .cΔMCOR irises or genes that are not expressed (13/24) in the iris of .cΔMCOR and .WT animals. Binding of SOX21 to Tgfβ2 was unambiguous (p<0.005) and strongly supported by JASPAR analysis which searches for a consensus SOX21-binding sequence in the 252 bp intronic region identified by CHIPseq (chr1:186,698,304-186,698,555; GRCm38/mm10 Assembly; 5.9 Kb downstream from the consensus donor splice-site of the 16 kb-long intron 1) (FIG. 2A). This sequence is conserved in the human TGFβ2 intron 1 orthologous region which comprises many potential transcription factors binding sites (GRCh37; chr1:218517865-218527740) (FIG. 2B)

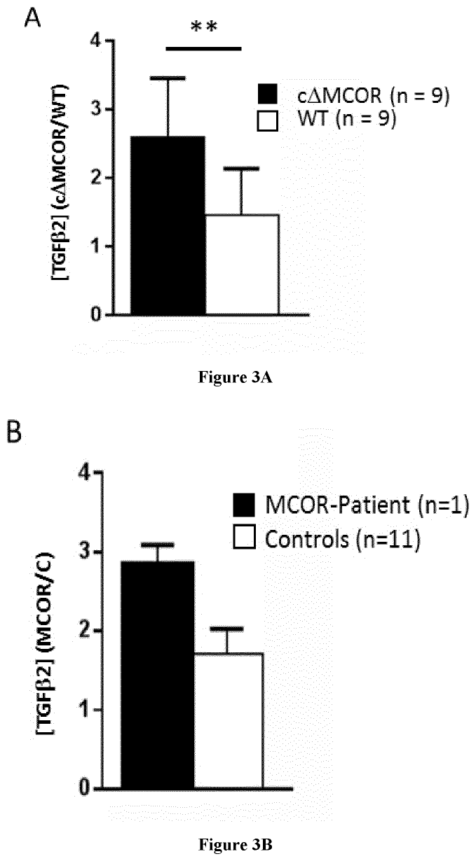

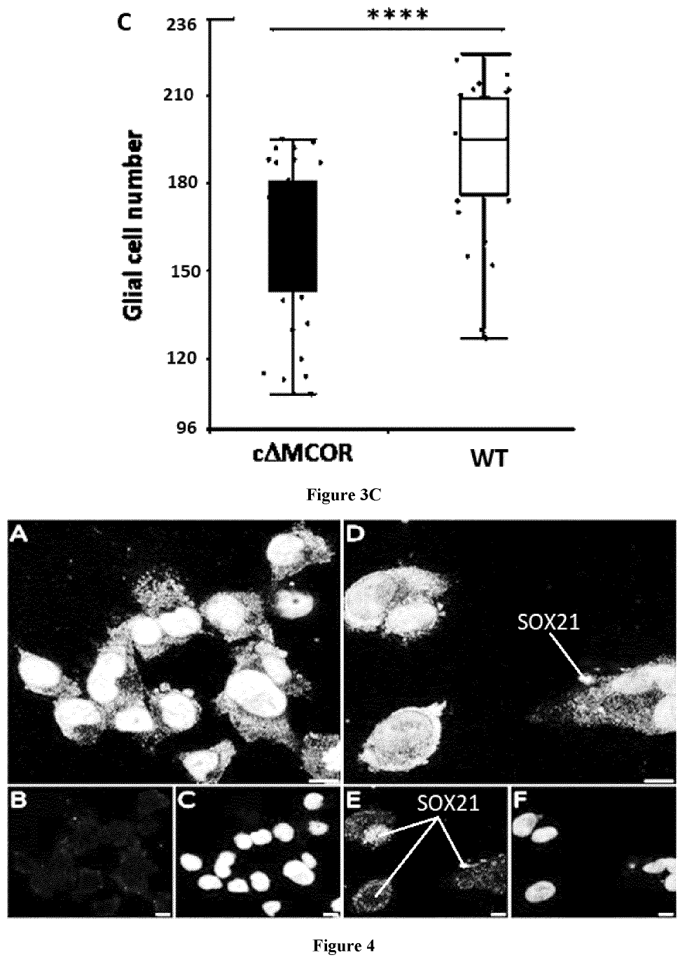

Many studies have reported significantly elevated levels of TGFβ2 in the aqueous humor of individuals with POAG (Agarwal et al., 2015; Wordinger et al., 2014) in cultured glaucomatous cell strains and isolated human glaucomatous TM tissues (Wordinger et al., 2014). The cause and cellular source of TGFβ2 accumulation in glaucomatous eyes is elusive, but it is clear that TM cells express an active TGFβ receptor complex and respond to exogenous TGFβ2, which increase extracellular matrix protein synthesis. Undue ECM synthesis in the TM increases resistance to aqueous outflow, leading to TOP elevation (Prendes et al., 2013). In human and mouse, high TOP initiates a cascade of events that result in a chronic and progressive deformation of the optic nerve head, a scenario that is observed as excavation or cupping of the optic disk (Quigley, 2011; Zeimer et al., 1998). The deformation of the ON head causes or contributes to the chronic degeneration of ON axons, and finally leads to apoptotic death of the retinal ganglion cells (RGC) (Munemasa & Kitaoka, 2013). Considering that (i) SOX21 is ectopically expressed in the CB where the aqueous humour is produced, (ii) SOX21 binds in a regulatory region of the Tgfβ2 gene and (iii) Tgfβ2 expression is upregulated in the iris of the MCOR mouse model, the inventors considered looking for TGFβ2 accumulation in the aqueous humor of .cΔMCOR mice. We confirmed accumulation by showing a significant increase of TGFβ2 concentration in the aqueous humor of mice carrying the critical MCOR deletion compared to WT counterparts (1.8 fold change, p<0.01; FIG. 3A). TGFβ2-mediated TOP increase and ECM accumulation in the TM of .cΔMCOR mice has not been analysed. But preliminary examination of the optic nerve head of one-year-old animals suggests loss of RGC axons, which is expected from the elevated concentration of TGFβ2 in the aqueous humor (FIG. 3C). Along the same line, the inventors had the rare opportunity to collect the aqueous humor of one non-glaucomatous adult MCOR individual of the Bretton family in the course of senile cataract surgery. Dosage of TGFβ2 concentration revealed a significant elevation as compared to 11 controls (all aqueous humor collected the same day, in the course of senile cataract surgery) (FIG. 3B).

To sum-up, by studying a mouse model of congenital microcoria, the inventors suggest that this ultra-rare and purely ocular disease is due to unanticipated complex mechanisms linked with 3D regulation of gene expression. We propose that the disease is due to the illegitimate expression of a transcription factor, SOX21, induced by the adoption of a DCT enhancer(s). We show that SOX21 binds to a regulatory region of the Tgfβ2 gene and the inventors demonstrate overexpression of this trophic factor in the iris and accumulation of its product in the aqueous humor of the mouse carrying the minimal MCOR deletion which recapitulates the observed accumulation in patients with POAG and one of our patient with MCOR. Consistent with studies which demonstrated a link between TGFβ2 accumulation in the aqueous humor and open angle GLC, our preliminary results indicate optic nerve degradation that is the hallmark of GLC, including POAG. Together, these observations further support the view that GLC in MCOR is not a consequence of the irido-corneal anomaly, but rather it seems to be a direct consequence of TGFβ2 overexpression as is POAG. Furthermore, knowing that TGFβ2 may act as a critical factor in axial elongation of the eye globe (Jia et al., 2017), its overexpression could also account for high myopia in MCOR. Finally, because SOX21 is not expressed in the iris anterior pigment epithelium, which gives rise to the dilator, the inventors propose that overexpression of TGFβ2 compromises the development of the dilator muscle by a paracrine signaling, which is consistent with the observation of high variability of histopathologic iris dilator muscle presentations reported in human individuals affected with MCOR. Thus, the inventors propose that overexpression of TGFβ2 links the iris malformation, myopia and GLC in congenital microcoria, making MCOR a highly valuable model to analyse eye development and the mechanisms of common POAG. Furthermore, our preliminary data disclose a novel pathway of TGFβ2 regulation which involves SOX21 as a potential therapeutic target for GLC both in MCOR and POAG.

### EXAMPLE 2: THE CRITICAL MCOR-CAUSING DELETION INDUCES SOX21 EXPRESSION IN HUMAN POSTERIOR EPITHELIAL CELLS OF THE IRIS

CRISPR-Cas9 RNA guides specific to the 5′ and 3′ boundaries of the 35 KB-critical MCOR-causing deletion in human 5′ gaggatatactaacaaagag 3′ (SEQ ID NO:49); 5′ gggagctgggcaggtaagaa 3′ (SEQ ID NO:50) were designed and cloned into pSpCas9(BB)-2A-GFP and pSpCas9(BB)-2A-mCherry plasmids, respectively.

SV40-immortalized human iris pigment epithelial cells (HIPEpiC) were co-transfected with the pSpCas9(BB)-2A-GFP and -mCherry plasmids encoding the RNA guides and double GFP/mCherry positive cells were sorted by flow cytometry, plated in culture well chambered coverglass and maintained in EPiCM culture medium (P60106, Innoprot; SV40-HIPEpiC) for 48 h to allow protein expression. Non-edited SV40-HIPEpiC and GFP/mcherry positive SV40-HIPEpiC were analyzed by immunocytochemistry using antibodies specific to the human SOX21 and DCT proteins (CL4688, Invitrogen and ab74073, Abcam), respectively. A positive DCT staining was observed both in non-edited and GFP/mcherry positive SV40-HIPEpiC cells. In contrast, while none of the non-edited SV40-HIPEpiC cells expressed SOX21, we observed a positive nuclear staining in 2% of co-transfected cells (FIG. 4).

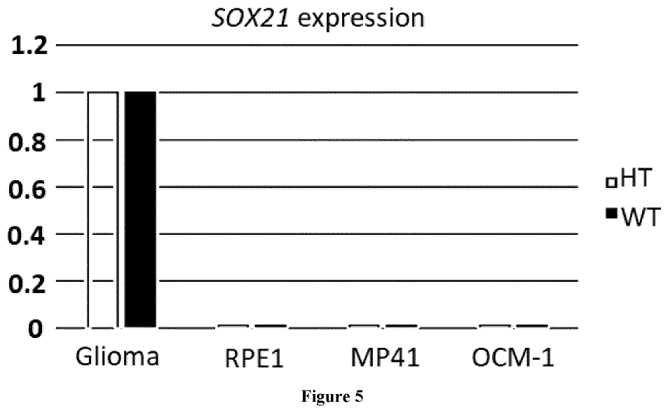

Telomerase-immortalized retinal pigment epithelium cells (RPE1) and human ocm-1, mp41 and U251 cells derived from ocular choroidal melanoma, uveal melanoma and glioma were edited and double GFP/mCherry positive cells were flow-sorted using the same strategy. Unique double GFP/mCherry positive RPE1, ocm-1, mp41 and U251 cells were plated to obtain clonal populations. Clones were analyzed for the presence of the critical deletion and SOX21 and DCT expression by Sanger sequencing of genomic DNA and RT-qPCR of mRNA, respectively. Both non-edited and edited glioma cells expressed DCT and SOX21 (positive control; FIG. 5). In contrast, non-edited and edited RPE1, ocm-1, mp41 lines expressed DCT but not SOX21.

Together, these data strongly support that the critical MCOR deletion cause ectopic expression of SOX21 in the posterior epithelium of the iris both in human and mouse.

### REFERENCES

Throughout this application, various references describe the state of the art to which this invention pertains. The disclosures of these references are hereby incorporated by reference into the present disclosure.
Agarwal, P., Daher, A. M., & Agarwal, R. (2015). Aqueous humor TGF-β2 levels in patients with open-angle. Molecular Vision, 9.
Aoki, T., Miyauchi, K., Urano, E., Ichikawa, R., & Komano, J. (2011). Protein transduction by pseudotyped lentivirus-like nanoparticles. Gene Therapy, 18(9), 936-941. https://doi.org/10.1038/gt.2011.38
Beckman, R. A., Weiner, L. M., & Davis, H. M. (2007). Antibody constructs in cancer therapy. Cancer, 109(2), 170-179. https://doi.org/10.1002/cncr.22402
Bonev, B., Mendelson Cohen, N., Szabo, Q., Fritsch, L., Papadopoulos, G. L., Lubling, Y., Xu, X., Lv, X., Hugnot, J.-P., Tanay, A., & Cavalli, G. (2017). Multiscale 3D Genome Rewiring during Mouse Neural Development. Cell, 171(3), 557-572.e24. https://doi.org/10.1016/j.cell.2017.09.043
Bremner, F. D., Houlden, H., & Smith, S. E. (2004). Genotypic and phenotypic heterogeneity in familial microcoria. The British Journal of Ophthalmology, 88(4), 469-473. https://doi.org/10.1136/bjo.2003.027169
Brummelkamp, T. R., Bernards, R., & Agami, R. (2002). A system for stable expression of short interfering RNAs in mammalian cells. Science (New York, N.Y.), 296(5567), 550-553. https://doi.org/10.1126/science.1068999
Carreon, T., van der Merwe, E., Fellman, R. L., Johnstone, M., & Bhattacharya, S. K. (2017). Aqueous outflow—A continuum from trabecular meshwork to episcleral veins. Progress in Retinal and Eye Research, 57, 108-133. https://doi.org/10.1016/j.preteyeres.2016.12.004
Checa-Casalengua, P., Jiang, C., Bravo-Osuna, I., Tucker, B. A., Molina-Martinez, I. T., Young, M. J., & Herrero-Vanrell, R. (2011). Retinal ganglion cells survival in a glaucoma model by GDNF/Vit E PLGA microspheres prepared according to a novel microencapsulation procedure. Journal of Controlled Release, 156(1), 92-100. https://doi.org/10.1016/j.jconrel.2011.06.023
Coulon, G., Delbosc, B., Jeffredo, Y., Viennet, G., Oppermann, A., & Royer, J. (1986). [Congenital microcoria: A case report with histopathological study]. Journal Francais D'ophtalmologie, 9(1), 35-39.
Davis, N., & Ashery-Padan, R. (2008). Iris development in vertebrates; genetic and molecular considerations. Brain research, 1192, 17-28. https://doi.org/10.1016/j.brainres.2007.03.043
Elbashir, S. M., Lendeckel, W., & Tuschl, T. (2001). RNA interference is mediated by 21- and 22-nucleotide RNAs. Genes & Development, 15(2), 188-200.
Fares-Taie, L., Gerber, S., Tawara, A., Ramirez-Miranda, A., Douet, J.-Y., Verdin, H., Guilloux, A., Zenteno, J. C., Kondo, H., Moisset, H., Passet, B., Yamamoto, K., Iwai, M., Tanaka, T., Nakamura, Y., Kimura, W., Bole-Feysot, C., Vilotte, M., Odent, S., . . . Rozet, J.-M. (2015). Submicroscopic Deletions at 13q32.1 Cause Congenital Microcoria. The American Journal of Human Genetics, 96(4), 631-639. https://doi.org/10.1016/j.ajhg.2015.01.014
Gould, D. B., Smith, R. S., & John, S. W. M. (2004). Anterior segment development relevant to glaucoma. International Journal of Developmental Biology, 48(8-9), 1015-1029. https://doi.org/10.1387/ijdb.041865dg
Hannon, G. J. (2002). RNA interference. Nature, 418(6894), 244-251. https://doi.org/10.1038/418244a
Holliger, P., & Hudson, P. J. (2005). Engineered antibody fragments and the rise of single domains. Nature Biotechnology, 23(9), 1126-1136. https://doi.org/10.1038/nbt1142
Holwerda, S. J. B., & de Laat, W. (2013). CTCF: The protein, the binding partners, the binding sites and their chromatin loops. Philosophical Transactions of the Royal Society B: Biological Sciences, 368(1620), 20120369. https://doi.org/10.1098/rstb.2012.0369
Iida, A., Tanaka, T., & Nakamura, Y. (2003). High-density SNP map of human ITR, a gene associated with vascular remodeling. Journal of Human Genetics, 48(4), 170-172. https://doi.org/10.1007/s10038-003-0002-x
Jia, Y., Yue, Y., Hu, D.-N., Chen, J.-L., & Zhou, J.-B. (2017). Human aqueous humor levels of transforming growth factor-β2: Association with matrix metalloproteinases/tissue inhibitors of matrix metalloproteinases. Biomedical Reports. https://doi.org/10.3892/br.2017.1004
Kabat, E. A., National Institutes of Health (U.S.), & Columbia University. (1991). Sequences of proteins of immunological interest. U.S. Dept. of Health and Human Services, Public Health Service, National Institutes of Health.
Kaczmarczyk, S. J., Sitaraman, K., Young, H. A., Hughes, S. H., & Chatterjee, D. K. (2011). Protein delivery using engineered virus-like particles. Proceedings of the National Academy of Sciences of the United States of America, 108(41), 16998-17003. https://doi.org/10.1073/pnas.1101874108
Kasetti, R. B., Maddineni, P., Millar, J. C., Clark, A. F., & Zode, G. S. (2017). Increased synthesis and deposition of extracellular matrix proteins leads to endoplasmic reticulum stress in the trabecular meshwork. Scientific Reports, 7(1). https://doi.org/10.1038/s41598-017-14938-0
Lan, X., Wen, L., Li, K., Liu, X., Luo, B., Chen, F., Xie, D., & Kung, H. (2011). Comparative analysis of duplicated sox21 genes in zebrafish. Development, Growth & Differentiation, 53(3), 347-356. https://doi.org/10.1111/j.1440-169X.2010.01239.x
Le Gall, F., Reusch, U., Little, M., & Kipriyanov, S. M. (2004). Effect of linker sequences between the antibody variable domains on the formation, stability and biological activity of a bispecific tandem diabody. Protein Engineering Design and Selection, 17(4), 357-366. https://doi.org/10.1093/protein/gzh039
Matsuda, S., Kuwako, K. -i., Okano, H. J., Tsutsumi, S., Aburatani, H., Saga, Y., Matsuzaki, Y., Akaike, A., Sugimoto, H., & Okano, H. (2012). Sox21 Promotes Hippocampal Adult Neurogenesis via the Transcriptional Repression of the Hes5 Gene. Journal of Neuroscience, 32(36), 12543-12557. https://doi.org/10.1523/JNEUROSCI.5803-11.2012
Mazzeo, V., Gaiba, G., & Rossi, A. (1986). Hereditary cases of congenital microcoria and goniodysgenesis. Ophthalmic Paediatrics and Genetics, 7(2), 121-125. https://doi.org/10.3109/13816818609076120
McBurney, S. P., Young, K. R., Nwaigwe, C. I., Soloff, A. C., Cole, K. S., & Ross, T. M. (2006). Lentivirus-like particles without reverse transcriptase elicit efficient immune responses. Current HIV Research, 4(4), 475-484. https://doi.org/10.2174/157016206778560018
McManus, M. T., Petersen, C. P., Haines, B. B., Chen, J., & Sharp, P. A. (2002). Gene silencing using micro-RNA designed hairpins. RNA (New York, N.Y.), 8(6), 842-850. https://doi.org/10.1017/s1355838202024032
Munemasa, Y., & Kitaoka, Y. (2013). Molecular mechanisms of retinal ganglion cell degeneration in glaucoma and future prospects for cell body and axonal protection. Frontiers in Cellular Neuroscience, 6. https://doi.org/10.3389/fncel.2012.00060
Muratori, C., Bona, R., & Federico, M. (2010). Lentivirus-based virus-like particles as a new protein delivery tool. Methods in Molecular Biology (Clifton, N.J.), 614, 111-124. https://doi.org/10.1007/978-1-60761-533-0_7
Pauls, S., Smith, S. F., & Elgar, G. (2012). Lens development depends on a pair of highly conserved Sox21 regulatory elements. Developmental Biology, 365-248(1), 310-318. https://doi.org/10.1016/j.ydbio.2012.02.025
Pozza, E., Verdin, H., Deconinck, H., Dheedene, A., Menten, B., De Baere, E., & Balikova, I. (2020). Microcoria due to first duplication of 13q32.1 including the GPR180 gene and maternal mosaicism. European Journal of Medical Genetics, 63(5), 103918. https://doi.org/10.1016/j.ejmg.2020.103918
Prendes, M. A., Harris, A., Wirostko, B. M., Gerber, A. L., & Siesky, B. (2013). The role of transforming growth factor β in glaucoma and the therapeutic implications. British Journal of Ophthalmology, 97(6), 680-686. https://doi.org/10.1136/bjophthalmol-2011-301132
Quigley, H. A. (2011). Glaucoma. Lancet (London, England), 377(9774), 1367-1377. https://doi.org/10.1016/S0140-6736(10)61423-7
Raisner, R., Kharbanda, S., Jin, L., Jeng, E., Chan, E., Merchant, M., Haverty, P. M., Bainer, R., Cheung, T., Arnott, D., Flynn, E. M., Romero, F. A., Magnuson, S., & Gascoigne, K. E. (2018). Enhancer Activity Requires CBP/P300 Bromodomain-Dependent Histone H3K27 Acetylation. Cell Reports, 24(7), 1722-1729. https://doi.org/10.1016/j.celrep.2018.07.041
Ramirez-Miranda, A., Paulin-Huerta, J. M., Chavez-Mondragón, E., Islas-de la Vega, G., & Rodriguez-Reyes, A. (2011). Ultrabiomicroscopic-Histopathologic Correlations in Individuals with Autosomal Dominant Congenital Microcoria: Three-Generation Family Report. Case Reports in Ophthalmology, 2(2), 160-165. https://doi.org/10.1159/000328751
Ramprasad, V. L., Sripriya, S., Ronnie, G., Nancarrow, D., Saxena, S., Hemamalini, A., Kumar, D., Vijaya, L., & Kumaramanickavel, G. (2005). Genetic homogeneity for inherited congenital microcoria loci in an Asian Indian pedigree. Molecular Vision, 7.
Reff, M. E., & Heard, C. (2001). A review of modifications to recombinant antibodies: Attempt to increase efficacy in oncology applications. Critical Reviews in Oncology/Hematology, 40(1), 25-35. https://doi.org/10.1016/s1040-8428(01)00132-9
Reiter, Y., Brinkmann, U., Lee, B., & Pastan, I. (1996). Engineering antibody Fv fragments for cancer detection and therapy: Bisulfide-stabilized Fv fragments. Nature Biotechnology, 14(10), 1239-1245. https://doi.org/10.1038/nbt1096-1239
Rouillac, C., Roche, O., Marchant, D., Bachner, L., Kobetz, A., Toulemont, P.-J., Orssaud, C., Urvoy, M., Odent, S., Le Marec, B., Abitbol, M., & Dufier, J.-L. (1998). Mapping of a Congenital Microcoria Locus to 13q31-q32. The American Journal of Human Genetics, 62(5), 1117-1122. https://doi.org/10.1086/301841
Sergouniotis, P. I., Ellingford, J. M., O'Sullivan, J., Fenerty, C. H., & Black, G. C. (2017). Genome sequencing identifies a large deletion at 13q32.1 as the cause of microcoria and childhood-onset glaucoma. Acta Ophthalmologica, 95(3), e249-e250. https://doi.org/10.1111/aos.13246
Simpson, W. A., & Parsons, M. A. (1989). The ultrastructural pathological features of congenital microcoria. A case report. Archives of Ophthalmology (Chicago, Ill.: 1960), 107(1), 99-102. https://doi.org/10.1001/archopht.1989.01070010101036
Tawara, A., Itou, K., Kubota, T., Harada, Y., Tou, N., & Hirose, N. (2005). Congenital Microcoria Associated With Late-Onset Developmental Glaucoma. Journal of Glaucoma, 14(5), 409-413. https://doi.org/10.1097/01.ijg.0000176931.29477.6e
Tham, Y.-C., Li, X., Wong, T. Y., Quigley, H. A., Aung, T., & Cheng, C.-Y. (2014). Global prevalence of glaucoma and projections of glaucoma burden through 2040: A systematic review and meta-analysis. Ophthalmology, 121(11), 2081-2090. https://doi.org/10.1016/j.ophtha.2014.05.013
Toulemont, P. J., Urvoy, M., Coscas, G., Lecallonnec, A., & Cuvilliers, A. F. (1995). Association of Congenital Microcoria with Myopia and Glaucoma. Ophthalmology, 102(2), 193-198. https://doi.org/10.1016/S0161-6420(95)31036-6
Tuschl, T., Zamore, P. D., Lehmann, R., Bartel, D. P., & Sharp, P. A. (1999). Targeted mRNA degradation by double-stranded RNA in vitro. Genes & Development, 13(24), 3191-3197. https://doi.org/10.1101/gad.13.24.3191
Uchikawa, M., Kamachi, Y., & Kondoh, H. (1999). Two distinct subgroups of Group B Sox genes for transcriptional activators and repressors: Their expression during embryonic organogenesis of the chicken. Mechanisms of Development, 84(1-2), 103-120. https://doi.org/10.1016/s0925-4773(99)00083-0
Vranka, J. A., Kelley, M. J., Acott, T. S., & Keller, K. E. (2015). Extracellular matrix in the trabecular meshwork: Intraocular pressure regulation and dysregulation in glaucoma. Experimental Eye Research, 133, 112-125. https://doi.org/10.1016/j.exer.2014.07.014
Weiss, A. H. (2003). Unilateral high myopia: Optical components, associated factors, and visual outcomes. British Journal of Ophthalmology, 87(8), 1025-1031. https://doi.org/10.1136/bjo.87.8.1025
Wordinger, R. J., Sharma, T., & Clark, A. F. (2014). The Role of TGF-β2 and Bone Morphogenetic Proteins in the Trabecular Meshwork and Glaucoma. Journal of Ocular Pharmacology and Therapeutics, 30(2-3), 154-162. https://doi.org/10.1089/jop.2013.0220
Young, N. M., MacKenzie, C. R., Narang, S. A., Oomen, R. P., & Baenziger, J. E. (1995). Thermal stabilization of a single-chain Fv antibody fragment by introduction of a disulphide bond. FEBS Letters, 377(2), 135-139. https://doi.org/10.1016/0014-5793(95)01325-3
Zapata, G., Ridgway, J. B. B., Mordenti, J., Osaka, G., Wong, W. L. T., Bennett, G. L., & Carter, P. (1995). Engineering linear F(ab′)2 fragments for efficient production in Escherichia coli and enhanced antiproliferative activity. Protein Engineering, Design and Selection, 8(10), 1057-1062. https://doi.org/10.1093/protein/8.10.1057
Zeimer, R., Asrani, S., Zou, S., Quigley, H., & Jampel, H. (1998). Quantitative detection of glaucomatous damage at the posterior pole by retinal thickness mapping. A pilot study. Ophthalmology, 105(2), 224-231. https://doi.org/10.1016/s0161-6420(98)92743-9
Zetsche, B., Gootenberg, J. S., Abudayyeh, O. O., Slaymaker, I. M., Makarova, K. S., Essletzbichler, P., Volz, S., Joung, J., van der Oost, J., Regev, A., Koonin, E. V., & Zhang, F. (2015). Cpf1 is a single RNA-guided endonuclease of a Class 2 CRISPR-Cas system. Cell, 163(3), 759-771. https://doi.org/10.1016/j.ce11.2015.09.038

1. A method for treating ocular disease in a subject in need thereof comprising administering to said subject a therapeutically amount of an inhibitor of SOX21 gene expression and/or activity.

2. The method according to claim 1, wherein the ocular disease is related to an increase of TGFβ2 expression and/or activity.

3. The method according to claim 1, wherein the ocular disease is selected from the group consisting of: Congenital microcoria (MCOR), glaucoma, open angle glaucoma (AOG, POAG) and myopia.

4. The method according to claim 1, wherein the inhibitor of SOX21 gene expression is siRNA, shRNA, miRNA, antisense oligonucleotide, a transcription factor decoy or a ribozyme.

5. The method according to claim 1, wherein the inhibitor of SOX21 activity is a peptide, polypeptide, peptidomimetic, small organic molecule, antibody or aptamers.

6. The method according to claim 1, wherein the inhibitor of SOX21 gene expression is delivered alone or in association with a viral vector.

7. The method of claim 6, wherein the viral vector is an adeno-associated virus (AAV) vector.

8. The method according to claim 7, wherein the viral vector is an AAV1, AAV2, AAV3, AAV4, AAV 5, AAV 6, AAV7, AAV 8 or AAV9.

9. The method according to claim 1, wherein the inhibitor of SOX21 gene expression and/or activity is delivered naked or with a viral vector and is delivered by intravitreous, subcutaneous, intravenous, ophthalmic drop or ophthalmic ointment delivery.

10. The method according to claim 1, wherein the inhibitor of SOX21 gene expression and/or activity is delivered naked or with a viral vector and is injected directly into the vitreous, aqueous humour, iris, ciliary body tissue(s) or cells and/or extra-ocular muscles, retina or suprachoridal space.

11. A pharmaceutical composition comprising an inhibitor of SOX21 expression and/or activity alone or in association with a viral vector.

12. (canceled)

13. (canceled)

14. The method according to claim 10, wherein the inhibitor of SOX21 gene expression and/or activity is delivered directly into the retina after retinal detachment.


SEQUENCE LISTING (50 sequences)

SEQ ID NO: 1 - DNA, 2924 bp, Homo sapiens
aaacactccagccgctgagagccccctttggcacttggcagcacgcggcggcgggctcctcggctcaacttcgaggagtctccgcgacgcaacttttggggacgctttgcatttaagagagaacgaccgaggaggaggagcgctctgcccggccgccgctacctgcggggagctcaccagcaaacgccactgcagacgaaggacccaaagaacgtaaagggcaaactgccgccgcggggagggggcaccgccgagaagttagagtgtcccagagacaacctgctcgagcgctcggccggagacactaaggcggcccggggcgcggcgtggccctgggctggtcccccagccccctcctccggggcgggagcgacgccggggcgcgacgagccccggccggccgagcgggtctccgcgggcagccaacattgatttcctccgggccgagggcgagggcccgggaggcggcgggctgcagccgcggcagggcgagagcatgtccaagccggtggaccacgtcaagcggcccatgaacgccttcatggtgtggtcgcgggctcagcggcgcaagatggcccaggagaaccccaagatgcacaactcggagatcagcaagcgcttgggcgccgagtggaaactgctcacagagtcggagaagcggccgttcatcgacgaggccaagcgtctacgcgccatgcacatgaaggagcaccccgactacaagtaccggccgcggcgcaagcccaagacgctgctcaagaaggacaagttcgccttcccggtgccctacggcctgggcggcgtggcggacgccgagcaccctgcgctcaaggcgggcgccgggctgcacgcgggggcgggcggcggcctggtgcctgagtcgctgctcgccaatcccgagaaggcggccgcggccgccgccgctgccgccgcacgcgtcttcttcccgcagtcggccgctgccgccgccgctgccgccgccgccgccgccgcgggcagcccctactcgctgctcgacctgggctccaaaatggcagagatctcgtcgtcctcgtccggcctcccgtacgcgtcgtcgctgggctacccgaccgcgggcgcgggcgccttccacggcgcggcggcggcggctgcagcggcggccgccgccgccggggggcacacgcactcgcaccccagcccgggcaacccgggctacatgatcccgtgcaactgcagcgcgtggcccagccccgggctgcagccgccgctcgcctacatcctgctgccgggcatgggcaagccccagctggacccctaccccgcggcctacgctgccgcgctatgaccccgcggggccgcctcgcgaggaccggtgtgcacacgtgtacatatgtataggtacgagcgctgcggcctccccgtgcgccctcccgcgaccgggggcccggtttgtatgtacatagaatgtataggtgccaggtagaggcagagaggccaggcggggcaggagtggccaagcgcgcaagggcgcgggcgagcaggcctgtgaattcgcaggatcatttcagacccgcacttcggcagccaactcgaaagcaggcggttgtgtgcggcagcagttggcgtttgctttgcacttcggaacctgttgcgttttgacccacggaggtggaggagtaactttttgacatgttggcctttccagttttgttggaagtttcatggtcggttttgtttttgtttctcattcttcttcctcgcccctcagccccccaacccccaaccccctcccggtccgtgttgcatgcacgctgttcaaatgtgaggtctgaaatggctggcacacgggaaaagctgcttgtgtcattcgtttctgggagtgggatggctctgagcagcctcgcctccctgtttgtactatttgaactttgcagatctctgttctctcaagcagaactcccaaccagatccattcttgaccagtgaccggctcgaatctggccttttgtgtgagatgatcacggtttcttttgtttatcacgccatttgcaaatcagagcaagagctctttctcaagggcaagaaacgcaaacaagaaatatttgtgagatgaaagttgtcaattggattttcttcctaaacaaacaacaacaacaaactactagaagtctccctgagtccactcgcttggatttctgacacagtttacaaaaaaggaaaaaggcactgctcctattttcccttatggctgagttcaccttaagattgtaaatgtgtatatgtcagtgaaaacattgaggcttggaaaatgtgttattttcgttgccctaagtttgagtcgactttagactcaaaaacattttgagcgaatatcaaagttaacttttaaaaattgcgaaactatttcagaatcgcaattttatcgaagattaaatcagacttttttgtctggtaattatatatttattatttagcaaaactgaagaaaaaaagcacagaattgtttcaacagatgtctctcattttcagctagcatttctctcccaagttgagctggtttaatgtgttttggatttccctcctcaattggcttattttttagatcacctgcaattcatttgcaaattgcaataaaacacattttagaaaaaaggaaccttcaattattagctttgtttctttttaaatgtatatattttgactaatgtttgtgaatgaagttggctaacatgtatttagtttcattttggctttatgtaatataaagtttttaaaattttaaatatggttttaacctttatgtgtaaatgattttctagtgtgaccttctaatttaatattagacgtctaaggtatatctgtaaattagaatccgactatcactctgttcattttttttgaacaaagagtttaaataaagcctgaaccagggaaaagaaaaa

SEQ ID NO: 2 - protein, 276 aa, Homo sapiens
MSKPVDHVKRPMNAFMVWSRAQRRKMAQENPKMHNSEISKRLGAEWKLLTESEKRPFIDEAKRLRAMHMKEHPDYKYRPRRKPKTLLKKDKFAFPVPYGLGGVADAEHPALKAGAGLHAGAGGGLVPESLLANPEKAAAAAAAAAARVFFPQSAAAAAAAAAAAAAGSPYSLLDLGSKMAEISSSSSGLPYASSLGYPTAGAGAFHGAAAAAAAAAAAAGGHTHSHPSPGNPGYMIPCNCSAWPSPGLQPPLAYILLPGMGKPQLDPYPAAYAAAL

SEQ ID NO: 3 - DNA, 17 bp, Mus musculus
aattcattgttctctgg

SEQ ID NO: 4 - DNA, 16 bp, Mus musculus
tgttattaaatttaaa

SEQ ID NO: 5 - DNA, 16 bp, Mus musculus
taaatttaaaataagt

SEQ ID NO: 6 - DNA, 16 bp, Mus musculus
tttaaatttaataaca

SEQ ID NO: 7 - DNA, 15 bp, Homo sapiens
aacaatgatagtttt

SEQ ID NO: 8 - DNA, 16 bp, Homo sapiens
tttcattttaaaaaat

SEQ ID NO: 9 - DNA, 17 bp, Homo sapiens
gctgttttgtttctttt

SEQ ID NO: 10 - DNA, 17 bp, Homo sapiens
gggcatttgtttatctc

SEQ ID NO: 11 - DNA, 17 bp, Homo sapiens
tgtcatttgtttggaat

SEQ ID NO: 12 - DNA, 16 bp, Homo sapiens
tttaataatatgttag

SEQ ID NO: 13 - DNA, 17 bp, Mus musculus
ccagagaacaatgaatt

SEQ ID NO: 14 - DNA, 16 bp, Mus musculus
tttaaatttaataaca

SEQ ID NO: 15 - DNA, 16 bp, Mus musculus
acttattttaaattta

SEQ ID NO: 16 - DNA, 16 bp, Mus musculus
tgttattaaatttaaa

SEQ ID NO: 17 - DNA, 15 bp, Homo sapiens
aaaactatcattgtt

SEQ ID NO: 18 - DNA, 16 bp, Homo sapiens
attttttaaaatgaaa

SEQ ID NO: 19 - DNA, 17 bp, Homo sapiens
aaaagaaacaaaacagc

SEQ ID NO: 20 - DNA, 17 bp, Homo sapiens
gagataaacaaatgccc

SEQ ID NO: 21 - DNA, 17 bp, Homo sapiens
attccaaacaaatgaca

SEQ ID NO: 22 - DNA, 16 bp, Homo sapiens
ctaacatattattaaa

SEQ ID NO: 23 - DNA, 23 bp, Artificial
  Synthetic c MCOR-guide RNA
ctcacagtttggtccaggctggg

SEQ ID NO: 24 - DNA, 20 bp, Artificial
  Synthetic CTCF1+Ps_guide RNA
tcttcagacgccgcgcttta

SEQ ID NO: 25 - DNA, 20 bp, Artificial
  Synthetic CTCF2-guide RNA
gtgttttatggacgggctcg

SEQ ID NO: 26 - DNA, 20 bp, Artificial
  Synthetic CTCF3_guide RNA
tccatagtaatgatcgcatc

SEQ ID NO: 27 - DNA, 20 bp, Artificial
  Synthetic Enh1-guide RNA
aacacagggaggtcgctttc

SEQ ID NO: 28 - DNA, 20 bp, Artificial
  Synthetic Enh2-guide RNA
tgggacacaagcaccggcct

SEQ ID NO: 29 - DNA, 23 bp, Artificial
  Synthetic c MCOR_guide RNA 3′
attccccagcagagaggcgctgg

SEQ ID NO: 30 - DNA, 20 bp, Artificial
  Synthetic CTCF1+Ps_guide RNA3′
gcccgctccgtttgctcgcc

SEQ ID NO: 31 - DNA, 20 bp, Artificial
  Synthetic CTCF2-guide RNA 3′
ctcggcataaagtttgtaat

SEQ ID NO: 32 - DNA, 20 bp, Artificial
  Synthetic CTCF3-guide RNA 3′
ctcggcataaagtttgtaat

SEQ ID NO: 33 - DNA, 20 bp, Artificial
  Synthetic Enh1_guide RNA 3′
caaaaatccttgggctaact

SEQ ID NO: 34 - DNA, 20 bp, Artificial
  Synthetic Enh2-guide RNA 3′
gttccagtagggcaacgcaa

SEQ ID NO: 35 - DNA, 23 bp, Artificial
  Synthetic primer 1
tgctcccctgttatgttcagatc

SEQ ID NO: 36 - DNA, 20 bp, Artificial
  Synthetic primer 2
gtgcaaaccaattcatgtta

SEQ ID NO: 37 - DNA, 20 bp, Artificial
  Synthetic SOX21 primer 1
gatgcacaactcggagatca

SEQ ID NO: 38 - DNA, 20 bp, Artificial
  Synthetic SOX21 primer 2
ggcgaacttgtcctttttga

SEQ ID NO: 39 - DNA, 20 bp, Artificial
  Synthetic DCT primer 1
aattcttcaaccggacatgc

SEQ ID NO: 40 - DNA, 20 bp, Artificial
  Synthetic DCT primer 2
ttgcgtggtgatcacgtagt

SEQ ID NO: 41 - DNA, 22 bp, Artificial
  Synthetic GusB primer 1
ctgcggttgtgatgtggtctgt

SEQ ID NO: 42 - DNA, 24 bp, Artificial
  Synthetic GusB primer 2
tgtgggtgatcagcgtcttaaagt

SEQ ID NO: 43 - DNA, 24 bp, Artificial
  Synthetic Hprt1 primer 1
gttggatacaggccagactttgtt

SEQ ID NO: 44 - DNA, 25 bp, Artificial
  Synthetic Hprt1 primer 2
aaacgtgattcaaatccctgaagta

SEQ ID NO: 45 - DNA, 25 bp, Artificial
  Synthetic Alb primer 1
gggacagtgagtacccagacatcta

SEQ ID NO: 46 - DNA, 22 bp, Artificial
  Synthetic Alb primer 2
ccagacttggtgttggatgctt

SEQ ID NO: 47 - DNA, 252 bp, Mus musculus
gatgtcttttaaacaaaagctgcaagagaatcttttggtctaaacctgacctagaacctgacctagaacctgacctagagcctgacctagagcctgacctagagcctgacctagaacctgacctagaacctgacctagagcctgacctagagcctgacctagagcctgacctagaaccctatcatgttattaaatttaaaataagtgcccagagaacaatgaatttataaatactatctctgggttaaatac

SEQ ID NO: 48 - DNA, 726 bp, Artificial
  Synthetic Human synthenic sequence sequences correspond to the murine and human
taggttcaggagtaaggtgagcagccgagcaagagactgcatggaagagctctggaaatagccccttccgctatcttgatgtagcatttccggtactgatgggctcccggagagaggatttggatttgccagatgtgcttgctctatttaggcagcgcttctgctcaagggtggattccattttgcagattctgcacagcttgatgaaatgtggctctgcccgagataaacaaatgcccattgtcattttgaaggcatatgtgtgctttcattttaaaaaatatcctttaaacaatgatagtttttaacctgggaattcaacgttctccaggtattttattttagatttaataatatgttaggggattctttgatgggtttgtttagactgaaaagtttgcttctgtcatttgtttggaataaatttgcatcatgctacaaccacaagctgcttttgatggttggctgttttgtttcttttctaagcgctgatatccagaagtgggtgtttaagtgccccacactctcaccactaccttacttcctgaattggcttggtggtcttgactctgaatgaaactgccccttggcctcttccctaccatctctaaattgccttcttttccttcattgtgatggaatgtctgcatggatggatggtcgagtgctcacatatcatgtcccatttgatactctactgagtttgtgggagacagcacccccttt

SEQ ID NO: 49 - DNA, 20 bp, Artificial
  Synthetic CRISPR-Cas9 guide primer 1
gaggatatactaacaaagag

SEQ ID NO: 50 - DNA, 20 bp, Artificial
  Synthetic CRISPR-Cas9 guide primer 2
gggagctgggcaggtaagaa